(19) United States
(12) Patent Application Publication
(10) Pub. No.: US 2021/0040435 A1
(43) Pub. Date: Feb. 11, 2021

(54) METHODS FOR AUTOMATED CONTROL OF A FERMENTATION SYSTEM

(71) Applicant: Culture Biosciences, Inc., South San Francisco, CA (US)

(72) Inventors: Matthew Adams BALL, Mountain View, CA (US); William Graham PATRICK, San Francisco, CA (US); Collin David James EDINGTON, San Mateo, CA (US)

(21) Appl. No.: 16/940,875

(22) Filed: Jul. 28, 2020

Related U.S. Application Data
(63) Continuation-in-part of application No. PCT/US2019/015437, filed on Jan. 28, 2019.
(60) Provisional application No. 62/623,239, filed on Jan. 29, 2018.
(60) Provisional application No. 62/668,458, filed on May 8, 2018.

(51) Int. Cl. C12M 1/36; H04L 29/08; B25J 11/00; B25J 9/16

(52) U.S. Cl. CPC C12M 41/48; H04L 67/10; B25J 9/0084; B25J 9/1669; B25J 11/00

(57) ABSTRACT

A system for controlling a fermentation system is described. The system can be controlled remotely by a user via cloud-based access to a computing platform connected to the fermentation system. A user can enter in an experimental protocol and make changes to the protocol in real-time.

### CROSS REFERENCE

This application is a continuation-in-part application of International Application No. PCT/US2019/015437, filed Jan. 28, 2019, which claims priority to U.S. Provisional Application No. 62/623,239, filed Jan. 29, 2018, and U.S. Provisional Application No. 62/668,458, filed May 8, 2018, each of which is incorporated herein by reference in its entirety.

### BACKGROUND

Large-scale fermentation processes can be used to produce healthcare products, food additives, alcohol, enzymes, biofuels, agricultural treatments, and industrial chemicals. During the fermentation process, microorganisms can be used to produce antibiotics, diagnostics, therapeutics, food products, chemicals, and biofuels. In the case of microbiome therapeutics or microbial agricultural treatment, the organisms themselves can be the product. Due to the importance of fermentation processes, an ability to control fermentation runs remotely and in an automated fashion could be valuable for the healthcare, food science, and biotechnology industries.

### INCORPORATION BY REFERENCE

Each patent, publication, and non-patent literature cited in the application is hereby incorporated by reference in its entirety as if each was incorporated by reference individually.

### SUMMARY

In some embodiments, the invention provides a computer program product comprising a computer-readable medium having computer-executable code encoded therein, which computer-executable code is configured to implement a method comprising providing an instruction for a fermentation system, wherein said computer program product is stored in a cloud-based system, wherein said computer program product is operably connected over said cloud-based system to said fermentation system, wherein the fermentation system comprises: a) a plurality of bioreactors configured to receive a fermentation agent, wherein at least one of the bioreactors of said plurality of bioreactors is removable and capable of having a different configuration from at least one other bioreactor of said plurality of bioreactors; and b) a first robotic component configured to provide said fermentation agent to at least one of said plurality of bioreactors.

In some embodiments, the invention provides a method for controlling a fermentation system, the method comprising: a) receiving by a computer program executable by a processor of a computer system an instruction for said fermentation system by a user, wherein said computer system is operably linked to said fermentation system, wherein said computer program is stored in a cloud-based system; and b) executing by said computer program executable by said processor of said computer system said instruction in response to input from said user; wherein said fermentation system comprises: i) a plurality of bioreactors configured to receive a fermentation agent, wherein at least one of the bioreactors of said plurality of bioreactors is removable and capable of having a different configuration from at least one other bioreactor of said plurality of bioreactors; and ii) a first robotic component configured to provide said fermentation agent to at least one of said plurality of bioreactors.

In some embodiments, the invention provides a system for controlling a fermentation system, the system comprising a computing system, wherein said computing system comprises a digital computer with access to a computing platform, wherein said digital computer comprises a computer processor and a computer memory comprising a computer program executable by said computer processor to generate an instruction for a fermentation system, wherein said computer program is stored in a cloud-based system, wherein said computer platform is operably linked to said fermentation system, wherein said fermentation system comprises: a) a plurality of bioreactors configured to receive a fermentation agent, wherein at least one of the bioreactors of said plurality of bioreactors is removable and capable of having a different configuration from at least one other bioreactor of said plurality of bioreactors; and b) a first robotic component configured to provide said fermentation agent to at least one of the plurality of bioreactors.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a schematic of a system described herein.

FIG. 2 provides an illustrative example of a system work cell described herein.

FIG. 3 depicts a detailed schematic of a system described herein.

FIG. 4 provides a computer control system for a fermentation system described herein.

FIG. 5 provides a computer control system for a fermentation system described herein.

FIG. 6 provides an illustrative network for use in a system described herein.

FIG. 7 provides an illustrative network for use in a system described herein.

FIG. 8 provides a zoomed-in view of an experimental design module of FIG. 7.

FIG. 9 provides a zoomed-in view of a protocol design module of FIG. 7.

FIG. 10 depicts an illustrative batch design user interface for selecting a microbial strain to be used in a system described herein.

FIG. 11 depicts an illustrative batch design user interface for selecting culture conditions to be used in a system described herein.

FIG. 12 depicts an illustrative fermentation process to be used in a system described herein.

FIG. 13 depicts an output to a user regarding availability of a microbial strain or culture medium to be used in a system described herein.

FIG. 14 depicts a fermentation protocol overview user interface.

FIG. 15 depicts an illustrative status report of currently running or previously run fermentation protocols run on a system described herein.

FIG. 16 depicts an internal check that can be performed by a system disclosed herein to determine whether the protocol is suitable for a system described herein, the duration of the run, and the cost of the run.

FIG. 17 provides an example of a graphical dashboard that could be displayed to a user during use of a system disclosed herein.

FIG. 18 shows an example of an electronics/communications architecture, in accordance with embodiments disclosed herein.

### DETAILED DESCRIPTION

The present disclosure provides an automated fermentation system that can be controlled remotely via a computing platform that is connected to the fermentation system. In some embodiments, the computing platform can be accessed via a cloud-based system. The present disclosure provides a fermentation system that can produce, for example, organisms and therapeutics in an efficient manner, while allowing a user to readily customize and modify fermentation protocols remotely.

### Automated Fermentation System

The present disclosure provides systems and methods for fermentation. Various aspects of the invention described herein can be applied to any of the particular applications set forth below. The invention can be applied as a fermentation automation work cell, or an integrated system for data collection and analysis. Different aspects of the invention can be appreciated individually, collectively, or in combination with each other.

An automated fermentation system disclosed herein can be of any size. For example, the automated fermentation system can be the size of a facility, a room, a car, a benchtop, or can be a handheld or portable system. The enclosure can enclose the space of a facility, a room, a car, a benchtop, or can be a handheld or easily transportable item. In some instances, the system can be larger than, approximately the same size as, or smaller than a shipping container. One or more dimensions of the system (for example, length, width, height, diagonal, diameter) can be less than or equal to about 1 cm, about 2 cm, about 3 cm, about 5 cm, about 10 cm, about 20 cm, about 50 cm, about 1 m, about 1.5 m, about 2 m, about 3 m, about 4 m, about 5 m, about 7 m, about 10 m, about 12 m, about 15 m, about 20 m, about 25 m, about 30 m, about 35 m, about 40 m, about 50 m, about 75 m, or about 100 m. One or more dimensions of the system can be greater than any of the values provided, or fall within a range between any two of the values provided. The enclosure can have one or more dimensions less than any of the values provided. One or more dimensions of the enclosure can be greater than any of the values provided or fall within a range between any two of the values provided. In some embodiments, a maximum dimension of the system or enclosure (greatest of length, width, or height) can have a value less than any of the values provided, greater than any of the values provided, or falling within a range between any two of the values provided.

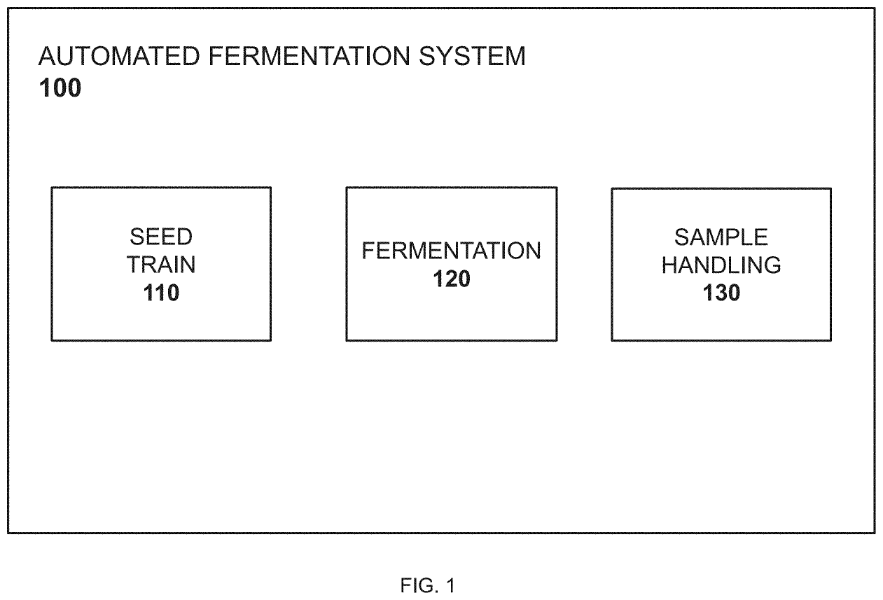

One or more processes within the automated fermentation system can be fully automated. One or more processes within the enclosure can be fully automated. A process can be automated and executed without requiring human intervention. A process can be automated when a human does not need to perform any manual manipulation. A process can be automated with the aid of one or more processors. A process can be automated if the presence of a human is not required within an enclosure of the automated fermentation system. In some embodiments, seed train preparation 110, fermentation 120, and/or sample handling 130 can be fully automated as shown in FIG. 1. In some embodiments, transfer of materials from a seed train station to a fermentation station can be fully automated. A seed train can refer to a process by which a sufficient number of fermentation agents are produced to inoculate the bioreactors. A seed train process can start with the thawing of a cryopreserved cell bank vial, followed by multiple culturing steps in progressively larger culture vessels.

In some embodiments, transfer of materials from a fermentation station to a sample handling station can be fully automated. In some embodiments, one or more preparation processes prior to fermentation can be automated. A fermentation process can be automated. Sample handling after fermentation can be automated. Sample handling can include sample preparation and/or analysis.

In some embodiments, one or more robotic components can aid in an automated process described herein. One or more robotic components can comprise one or more robotic arms. A description herein of a robotic arm can apply to any type of robot or robotic component. For example, any description of an arm can apply to a gantry, such as a three-axis gantry. A robotic arm can be capable of interacting with a seed train preparation station, a fermentation station, and/or a sample handling station. A robotic arm can aid in transfer of materials within a seed train preparation station, within a fermentation station, and/or within a sample handling station. A robotic arm can be capable of aiding in the transfer of materials between a seed train preparation station and a fermentation station, or between a fermentation station and a sample handling station.

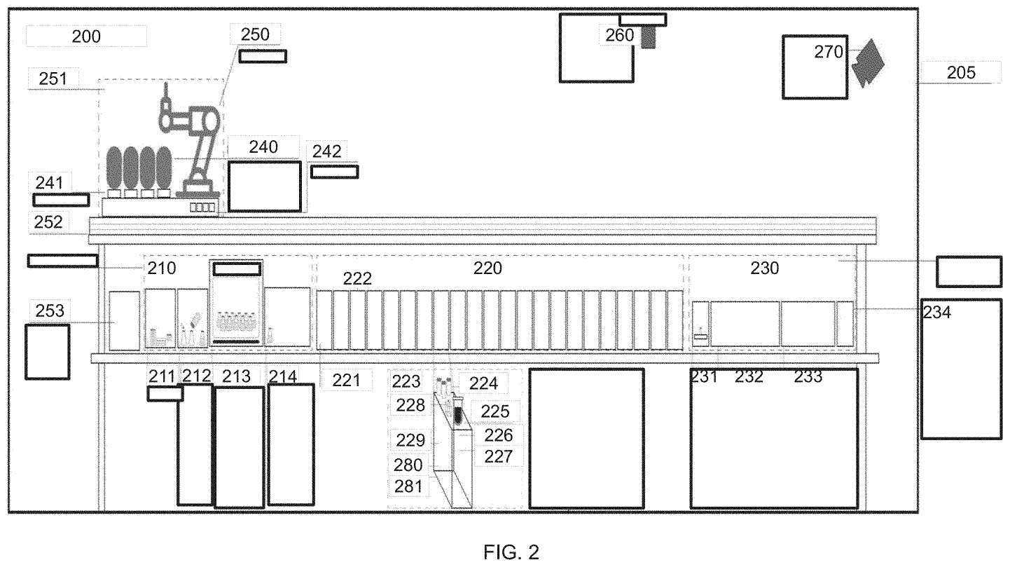

A work cell 200 can comprise one or more robots 250 as shown in FIG. 2. A description here of a robot can apply to a robot arm 250, and vice versa. A description of a robot can comprise one or more robotic components capable of actuation. A robot can comprise a robot arm 250. The robot arm can be a 6-axis robot arm. The robot arm can be capable of motion about 1 or more, two or more, three or more, four or more, five or more, or six or more axes of motion. The robot arm can comprise one or more, two or more, three or more, four or more, five or more, six or more, seven or more, eight or more, nine or more, or ten or more joints. The joints can comprise motors that can allow various support members to move relative to one another. The robot arm can comprise one or more, two or more, three or more, four or more, five or more, six or more, seven or more, eight or more, nine or more, or ten or more support members. In one example, a first support member can bear weight of an end effector. A second support member can bear weight of the first support member and/or the end effector, and so forth. The motors can allow rotation of one or more support members relative to one another. One or more sliding mechanism can be provided that can allow lateral displacement. One or more telescoping components for support members can or cannot be provided. The robot arm can have a free range of motion that can match or exceed the range of motion of a human arm. Ball and socket joints can or cannot be employed by the robot arm.

The robot can have any dimensions. In some instances, a dimension (e.g., length, width, height, diagonal, diameter) can be at least about 1 cm, about 3 cm, about 5 cm, about 7 cm, about 10 cm, about 15 cm, about 20 cm, about 30 cm, about 40 cm, about 50 cm, about 60 cm, about 70 cm, about 80 cm, about 90 cm, about 1 m, about 1.2 m, about 1.5 m, about 1.7 m, about 2 m, about 2.5 m, or about 3 m. The dimension can be less than any of the values provided or can fall within a range between any two of the values provided. The robot can have a maximum dimension that is less than any of the values provided herein, greater than any of the values provided herein, or falling within a range between any two of the values provided herein.

The robot can comprise a robot carriage 251. In some embodiments, a robot arm can be supported on a robot carriage. The robot carriage can bear weight of the robot arm. The robot carriage can support the robot arm. The robot carriage can support a robot arm on a top surface of the robot carriage, a bottom surface of the robot carriage, and/or a side surface of the robot carriage. A robot carriage can support a single robot arm or multiple robot arms. One or more robot arms can be affixed to the carriage or can be movable relative to the robot carriage at the location where the robot is supported by the robot carriage. Robot arms supported by the robot carriage can have the same characteristics or can have one or more differing characteristics (for example, size, number/type/direction of joints, number/type/characteristics of support members, end effectors, or materials).

The robot carriage can be capable of motion. The robot carriage can move relative to the rest of the work cell. The robot carriage can move relative to one or more bioreactors 221. The robot carriage can move relative to equipment used for seed train preparation, and/or sample handling. The robot carriage can be supported be a support mount, such as a linear rail 252. The robot carriage can move in a translational manner along the support mount. For instance, the robot carriage can move laterally and/or vertically along a support mount. The support mount can comprise one or more straight lines, curves, and/or corners. The support mount can be formed from a single track or can comprise multiple tracks that the robot carriage can follow. A support mount can be elevated. The support mount can be supported by a work cell floor, wall, and/or ceiling. In some instances, a location of a robot can be measure and/or monitored with aid of the support mount. In some instances, the support mount can have a known location and the location of the robot carriage relative to the support mount can be determined. In some embodiments, one or more motors and/or sensors can be provided on a support mount, such as a linear rail, to effect movement of the robot carriage. Optionally, one or more motors and/or sensors can be provided on a robot carriage to effect movement of the robot carriage.

In some instances, the robot carriage can be capable of movement without being restricted to a track or rail. The robot carriage can move autonomously or semi-autonomously. In some instances, the robot carriage can move across a surface. For example, one or more sets of wheels, legs, arms, treads, gliders, or other components can be used to propel a robot carriage. A robot carriage can drive along a floor of a work cell. A robot can be supported by a quadcopter or another type of flying vehicle or drone. A robot can be capable of flight within a work cell.

The robot carriage can optionally bear weight of one or more bulk media containers 240. The robot carriage can or cannot support one or more bulk media containers. One or more bulk media containers can move with the robot carriage. For instance, if a robot carriage navigates a rail 252, the bulk media containers can move along with the robot carriage along the rail.

A location and/or position of the robot can be monitored. In some embodiments, one or more sensors on a robot arm, robot carriage, support mount, or other portion of the work cell can be used to determine the location of the robot within the work cell and position of one or more components of the robot. This can be useful when the robot needs to execute precise motions in interacting with various components of the work cell. In one example, servomotors can be employed that can be useful for determining position, speed, or acceleration of the robot, or one or more components of the robot.

In one example, a robot can comprise an end effector 253. For instance, one or more end effectors can be positioned at an end of a robot arm. In some instances, end effectors can be provided at other locations along a robot arm. An end effector can interact with one or more other components of a work cell. For instance, an end effector can manipulate or interact with one or more containers or equipment. An end effector can be used to lift and/or transport a container. An end effector can be used to rotate or flip a container. An end effector can be used to interact with equipment (for example, press a button, flip a switch, turn a dial, open/close a door, or touch a touchscreen).

Various types of end effectors can be employed. In one example, an end effector comprises a gripper. A gripper can grasp one or more objects. A gripper can comprise two or more ‘fingers’ that can be capable of movement relative to one another. A gripper can be moved relative to the rest of the arm and allow an object held by the gripper to move rotationally and/or translationally.

In some embodiments, an end effector can utilize magnets, vacuum suction, fasteners, cutters, sensors (for example, cameras, barcode readers, or microphones), emitters (for example, light or sound), or other components to sense and/or interact with other components of the work cell. In one example, an end effector can comprise a pipettor, for example, to provide liquid handling capabilities. In another example, an end effector can comprise an optical detector, such as a camera or barcode reader. Different types of end effectors can be provided. In some instances, multiple of the same type of end effectors can be provided. They can have the same dimensions or other characteristics, or different dimensions or other characteristics.

An end effector can move in any direction. For instance, an end effector supported by a robotic arm, can translate along one or more, two or more, or three or more axes, or can rotate about one or more, two or more, or three or more axes. An end effector can rotate about a roll axis, pitch axis, and/or yaw axis.

In some embodiments, multiple types of end effectors can be utilized by a robot. The end effectors can be swappable 253. For example, a first end effector can be removed from a robot arm. A second end effector can then be attached from the robot arm. The first end effector and the second end effector can be of the same type or different types. The first end effector and the second end effectors can have the same characteristics or can have at least one characteristic that is different. In some instances, a robot arm can utilize a single end effector at a time. Alternatively, a robot arm can be capable of utilizing multiple end effectors at a time. A work cell can have one or more locations where end effectors that are not being used by the robot are stored. The robot can drop off and/or pick up new end effectors as needed. The robot can swap end effectors according to need. In some embodiments, a work cell can comprise multiple robots. The multiple robots can share the same pool of end effectors. Alternatively, each robot can have its own set of end effectors that it can access.

Any number of robots can be selected for a work cell. In some embodiments, a single robot can be provided for a work cell. In other instances, multiple robots can be selected for a work cell. In some embodiments, the number of robots can depend on a number of bioreactors at a fermentation station. For example, at least one robot can be provided for each at least 1 bioreactor, 2 bioreactors, 3 bioreactors, 4 bioreactors, 6 bioreactors, 8 bioreactors, 10 bioreactors, 12 bioreactors, 18 bioreactors, 24 bioreactors, 36 bioreactors, 48 bioreactors, 60 bioreactors, or 96 bioreactors. In some instances, selected robots can have selected roles and not perform other roles that performed by other robots. In some instances, each robot can be capable of performing any role within the work cell. In some embodiments, a selected robot can only interact with selected bioreactors while not interacting with other bioreactors. For instance, if two robots are provided, a first robot can interact with bioreactors 1-12 while a second robot can interact with bioreactors 13-24. When two or more robots are provided, then the two or more robots can be directed to interact with non-sequential bioreactors, for example, the first robot can interact with bioreactor 1, 3, 5, and 7, and the second robot can interact with bioreactors 2, 4, 6, and 8.

Alternatively, any of the robots in the work cell can interact with any of the bioreactors on an as-needed basis. For instance, if two robots are provided, both the first and second robots can be capable of interacting with any of bioreactors 1-24. In another example, selected robots can only interact with certain stations or sets of equipment. For instance, a selected robot can interact with a seed train station while not interacting with the sample handling station. Alternatively, any of the robots can interact with any of the stations or any of the equipment.

The bioreactors can also be capable of motion. The bioreactors can be mobile and take themselves to stationary robotic arms, or a robotic system could transport the bioreactors to a fixed robotic arm.

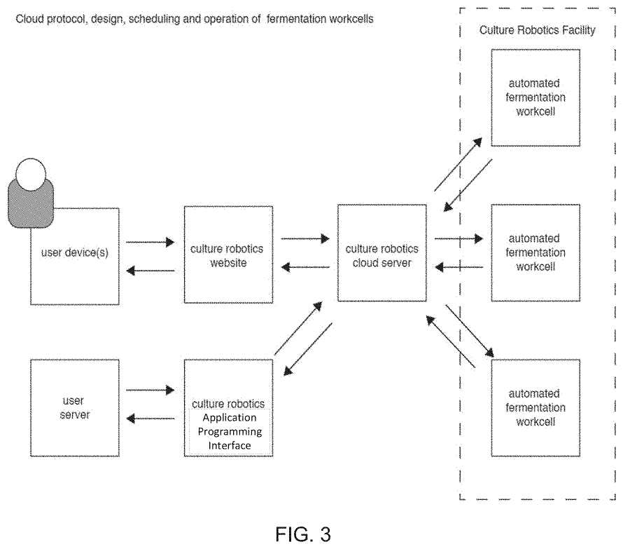

FIG. 3 illustrates a high-level work flow of a system of the present disclosure. FIG. 3 shows that a user can access a system website from a user device. From the website, the user can enter in the desired experimental protocol, which can be communicated to the server of the system, which can be a cloud-based server. Based on the entered experimental protocol, an automated fermentation work cell can begin the fermentation process. An alternative path for accessing the system server can be through a user server, which can be operably linked to the application programming interface (API) of the system without the need for a website. After the user has entered in the desired experimental protocol into the API of the system, the experimental protocol can be communicated to the cloud-based server of the system, allowing the fermentation process to begin.

A fermentation system described herein can contain a work cell that can be automated and programmable and controlled by a cloud interface. A user of the system can design an experiment on, for example, a web interface that can automatically be checked by a software system described herein. An experiment designed by the user can be composed of, for example, a fermentation agent, such as a microbial strain, a fermentable substrate, such as a culture medium for the fermentation agent, and a fermentation protocol.

The user can define the fermentable substrate and fermentation agent to be used in a fermentation protocol. The user can select pre-defined protocols that are part of a database of the system, or the user can customize a protocol. The software system of the present disclosure can check for any configurations needed in the hardware based on the proposed experimental design. The user can view the progress of the fermentation protocol when the work cell is open and then schedule runs. The software of the present system can view the desired experiment and the existing demand, and then calculate a cost/bioreactor-run for the user. The experimental protocol and schedule provided by the user can be turned into a set of instructions for the fermentation system. The present software can turn the user-defined experimental protocol into an operator-robotic program.

During a run, data can come into the cloud system from many sources in and out of the work cell including, for example, data from a bioreactor within the work cell, data from pieces of analytical equipment that can be automatically run inside the work cell, data from software or computer vision-based sensors that can track the movement of operators coming in and out of the work cell, and temperature sensors that can monitor the ambient condition of the work cell or the surrounding environment.

During a run, a user can view the live data from, for example, the work cell and images from the experiment, and then make real-time changes to the protocol, which could include changing protocol set points or changing the entire protocol to a new protocol. Additionally, the user can stop or pause the run based on the data feed. Based on the data coming into the system, the cloud system of the present disclosure can make live estimations of important parameters during the experiment. After a run, the user can view the data from the experiment in the protocol. An API can also allow a user to design a protocol, schedule an experiment, ingest live data, and control the experiment.

### Planning Module

The present disclosure provides a fermentation system that can be controlled via, for example, a user-provided experimental protocol. Based on the experimental design, the fermentation work cell can be automatically programmed into a series of steps that can be completed by either a robot or a human operator. An experimental protocol could be provided by, for example, a user, a customer, an operator, or from an open source protocol. Additionally, an experimental design or protocol can be created a priori by an automated system disclosed herein, or the protocol can be designed and modified during a fermentation run.

A fermentation system described herein can comprise a planning module. The series of steps of an experimental protocol can be part of a planning module for the fermentation system. The steps can be prioritized and can be placed in a common queue. Steps can be interrupted or re-ordered depending on the state of the queue. The queue can be truncated. The queue can be redistributed among additional automated systems (e.g. robotic arms) or personnel. A task can switch from an automated task to a manual task depending on the state of the queue, the presence of other tasks, the state of automated systems and/or the state of personnel.

The planning module can be a software compiler, in which the planning module can take a high-level experimental design and divide the high-level experimental design into a series of low-level tasks that can be completed by the robot and/or a human operator. The planning module can comprise a software planner. The planning module can translate protocol steps into lower level actuation and robotic moves. For example, the planning module can translate an experimental protocol wherein the protocol requests that a sample be taken at hour 47 at bay 12 into a set of robotic steps, which can include: (1) bringing a sample tube over to bay 12 at hour 47; (2) actuating a pump on the bay 12 reactor to pump out a sample; (3) removing the sample tube and placing the sample tube in a cold storage container; and (4) alerting a human operator to collect that sample or a set of samples at a certain time.

The planning module can compile steps, also known as robotic paths, in an efficient manner. The step or path planning algorithm can make all the robotic steps occur at a time defined in the experimental protocol. During an experiment, the software planner can track the steps as the steps are being executed by the work cell. If the work cell gets stuck on a task or has an error during the experiment, then the software planner can alert a user, and the user can modify the experimental protocol in real-time, as needed. The error in the work cell can be determined by sensors that are place inside and around the work cell. For example, a sensor placed in the work cell can determine that the work cell collected a 0.25 mL sample instead of a 2 mL sample, as specified in the work cell. If a sampling error occurs, then the fermentation system can repeat the sampling step.

During operation of an experimental protocol, the planning module can incorporate new steps from a user or an operator. For example, if the user wants to plan a new sample for one of the work cells, then the planning module can incorporate that direction, schedule a new robotic task to take a sample, and schedule a task for a user to pick up the sample. The planning module can also ensure that there is enough time to insert a new task into the experimental protocol where desired, based on the availability of the robot and whether a technician would be available at that time.

The planning module can create a standard operating procedure or set of tasks to be completed by the user throughout the experiment. These tasks can be available on, for example, a mobile device and the operator can see what tasks are required and when the tasks need to be executed.

A user can use a system described herein for large-scale fermentation processes, for use in, for example, industrial applications. In a large-scale manufacturing system, parameters such as, for example, rates of cooling, pumping, mixing, gas addition, concentrations of input liquids, gas composition, media composition can be adjusted to accommodate large-scale fermentation processes. Certain timelines can be adjusted with respect to sampling, inoculation and liquid addition.

When configured to do so, the planning module can recommend or disallow certain parameters that would be favorable during large-scale fermentation. The planning module can indicate when certain parameters are required to achieve large-scale fermentation. The scale-up factors can be based on, for example, volume, vessel geometry, shear rates, oxygen transfer data or other parameters.

Real manufacturing facilities can be captured in the software tool and the planning module can reference against these values. This data can be uploaded by other facilities via an automated programming connection or by an end user. End users can evaluate their protocols against these parameters from real facilities or against other custom values. Scale-up data from past experiments can also be presented by the software. The data can be used by the software or by end users to determine if a similar process will effectively scale-up to manufacturing level.

The planning module can suggest adjustments to the experimental protocol that can achieve a given goal without creating a condition that cannot scale up. For example, if the goal is to produce a certain quantity of product, the planning module can recommend adjusting the run time of the experiment, given a reduction in oxygen transfer rates at large scale.

### Configuration Module

A fermentation system described herein can further comprise a configuration module. The configuration module can be configured to perform automated hardware configuration of the work cell hardware based on the experimental protocol. An executed protocol can begin with hardware configuration of the work cell. The fermentation system can check whether the necessary hardware is already in the work cell. If the necessary hardware is not in the work cell, then the planning module can create a task for a user to add any necessary hardware. Some steps of the experimental protocol can require feedback loops to know if the steps have been completed. A planning module of the fermentation system described herein can either be locally in a computer operably connected to the automated work cell or can be part of a cloud-based server.

Based on the experimental protocol designed by the user, hardware configuration can be performed in the reactors of the work cell. The planning module can check for the hardware required for the protocol and compare to the current hardware configuration in the work cell. If the work cell requires hardware changes, then the planning module can create a list of tasks for an operator to make the necessary changes to the hardware. Before the experiment, the planning module can check that all of the required hardware configurations have been made and that the work cell is ready for the experimental protocol. The configuration module can allow the workcell to perform a subset of normal tasks if certain hardware is not present. The configuration module can allow tasks to be delayed (and then re-queued) when the requisite hardware modules are present.

### Experimental Design Module

An experimental design module of the present system can allow for online experimental design by, for example, a user, a customer, or an operator. The experimental design module can include steps that would require different parts of the work cell to move and operate to execute the desired experiment. An experiment provided by the user can contain one or multiple bioreactor-runs. Each bioreactor-run or experiment can be composed of, for example, the fermentation agent being tested, the fermentable substrate or the set of medium being used, and the fermentation protocol being used.

For the set of fermentation agents, the user can define the fermentation agent by giving the fermentation agent a name, or the name can be assigned (for example, by a supplier), for example, by scanning a barcode. The name can be specific in terms of the class and subclass of the fermentation agent, and can specify the base species in-use and the replica class. A replica class can be created using a replica plating technique. Replica plating is a method by which each colony/clone is inoculated onto multiple plates according to a numbered scheme, which allows each clone to be tested by a variety of methods, while retaining a master plate from which clones can be picked.

The user can specify the handling instructions for the fermentation agents. For example, the user can specific how the fermentation agent will be labeled and stored during transfer and how the fermentation agent should be amplified (grown) before the fermentation process begins.

For the fermentable substrate, the user can name each of the fermentable substrates that can be used in the experiment. The user can define, for example, the concentration of the fermentable substrate and various components therein. The user can define the correct handling conditions for each fermentable substrate. The user can specify the manufacturer and part number for each fermentable substrate. The user can also specify suitable alternatives for fermentable substrates. The user can specify the class and subclass of each fermentable substrate (e.g. carbon source: feed, or base control: NaOH). The user can specify the molecular formula, molecular mass, and volume of each fermentable substrate.

A user can select a protocol that is part of the database of a software system described herein, or provide a customized protocol.

The experimental design can be split into two major categories: strain screening and process development. In strain screening, a single strain screening protocol can be used to test a number of different fermentation agents. In process development, one fermentation agent can be used, but the protocol and/or medium can be varied. For example, process parameters, such as temperature, pH, dissolved oxygen set-points, feeding rates, and medium composition can be tested to determine the optimal conditions for the selected fermentation agent. Control strategies for temperature, pH, dissolved oxygen, substrate feeding, antifoam addition and induction addition can be specified. The user can define when the fermentation agent and the fermentable substrate should be picked up by the fermentation system. The hardware configuration may also be varied in an experiment.

### Protocol Design Module

A protocol design module of the present system can allow for online protocol design by a user. The protocol design module can include steps that would require different parts of the work cell to move and operate to execute the desired protocol. The user can enter the protocol in, for example, a graphical and/or programming interface on the web. An experiment provided by the user can contain one or multiple bioreactor-runs. Each bioreactor-run or experiment can be composed of, for example, the fermentation agent being tested, the fermentable substrate or the set of medium being used, the fermentation protocol being used, a series of set-points that can be constant through an experiment or time variable, and logic that can control bioreactor pumps, agitation motors, heating and cooling systems, and mass flow controllers based on sensor readings that are part of the fermentation system. The user can specify a set of run-time media additions by time, volume, type, and concentration. The user can specify times and volumes for run-time sampling. The protocol may be serialized and then imported (or exported) using a common file format such as DAT, TXT, XLSX, JSON, YAML, TOML.

The set of inputs to control logic can include, for example, the sensors in a bioreactor (for example, pH, dissolved oxygen, temperature, and biomass), offline (ones that are outside the reactor) analytical tools that can obtain data from liquid broth or gas samples (including spectrophotometers, biochemical analysis, and off-gas analysis), and any software sensors, which can include, for example, the volume of the reactor estimated by the flow rates of pumps, an estimate of the cell concentration or metabolism (which can be derived from multiple inputs such as the amount of cooling being performed to maintain the temperature of the bioreactor and the imagery of the bioreactor), the amount of oxygen (or other gas) being transferred to the broth from the inlet gas, the amount of oxygen consumed by the fermentation agents, or the amount of carbon dioxide being produced by the fermentation agents, and an estimate of the carbon substrate in the reactor.

The inputs to control logic can also include the performance of bioreactors that are running in real-time or that have run previously. The inputs can also include a modeled future state of the bioreactor, including any process parameters, and forecast out to a duration of anywhere between, for example, 0.001 seconds, 0.01 seconds, 0.1 seconds, 1 second, 5 seconds, 15, seconds, 30 seconds, 45 seconds, 1 minute, 5 minutes, 10 minutes, 15 minutes, 20 minutes, 25 minutes, 30 minutes, 1 hour, 2 hours, 3 hours, 4 hours, 5 hours, 6 hours, 12 hours, 1 day, 2 days, 3 days, 4 days, 5 days, 6 days, 1 week, 2 weeks, 3 weeks, 1 month, 2 months, 3 months, 6 months, or 1 year.

The heating/cooling system, all of the feed pumps (for example, substrate feed control, acid/base), the impeller RPM (revolutions per minute), and the volumetric flow rate of gasses into the system can further by controlled by a protocol provided by a user. The user can also specify how samples are handled after their collection. Samples can be stored at, for example, −80° C., −20° C., 4° C., 25° C., or any range of temperatures within. The samples can be treated with a chemical agent to arrest growth or to freeze metabolic activity. The samples can be prepared for processing by an analytical instrument, the samples can be treated with a chemical to cause cell lysis, the samples can be centrifuged, or the samples can be aliquoted. All of the foregoing sample handling procedures can be specified by a user. A user can also specify the sample storage vessel.

Per-sample protocols may also be specified. These protocols can be created de-novo or used from a library of other protocols.

The protocol can also include a set of steps for the seed train, medium preparation, feeding, and sample preparation and storage. The overall length of the experiment can also controlled by a protocol provided by the user in the protocol design module. Endpoints of the protocol can be, for example, time, cell mass, or titer/production-based. The number of samples and types of analyses can also be controlled by the protocol provided by the user in the protocol design module.

The endpoints of a protocol can be triggered manually by a user. The endpoints can be dynamically affected by any online or offline measurements on each reactor or a plurality of reactors, or by a user's decision.

The protocol design module can further allow end users to review their protocols and experimental data in a collaborative manner. Users can leave comments and suggestions for improvements. A stream of comments can be viewed from a variety of users of the system. The comments can be edited, deleted, and linked-to for further reference. The suggestions in the comments can be accepted, rejected, or deleted by users of the system. The comments and suggestions can be hidden to certain users depending on their level of access or permissions, and commenting and suggesting can be restricted to certain end users. The protocol design module can be enabled to accept comments and suggestions. The protocol design module can be disabled to reject comments and suggestions.

### Monitoring Module

A monitoring module of the present system can perform automatic checking and validation of experiments and protocols entered by a user. A monitoring module may also be referred to as validation module which can be used interchangeably throughout the specification. Based on the automatic checking protocol, the system can determine whether the hardware of the present system can hit the desired parameters and controls provided by a user, and alert a user if the system can or cannot hit these parameters.

The validation module can check a protocol by, for example, hardcoding a set of limits, such as a temperature set-point. The validation module can further learn from previous experiments how quickly the present control system can respond to various parameter changes during a fermentation run. The checking protocol can validate that the protocol will end and will not use any methods incorrectly. The checking protocol can check for typographical errors or logical errors. The checking protocol can standardize the format of the protocol for simplified sharing. The checking protocol can make recommended changes based on past modifications made by the same customer. The checking protocol can make recommended changes based on industry-standard practices.

The validation module can further allow end users to review their protocols and experimental data in a collaborative manner. Users can leave comments and suggestions for improvements. A stream of comments can be viewed from a variety of users of the system. The comments can be edited, deleted, and linked-to for further reference. The suggestions in the comments can be accepted, rejected, or deleted by users of the system. The comments and suggestions can be hidden to certain users depending on their level of access or permissions, and commenting and suggesting can be restricted to certain end users.

### Display Module

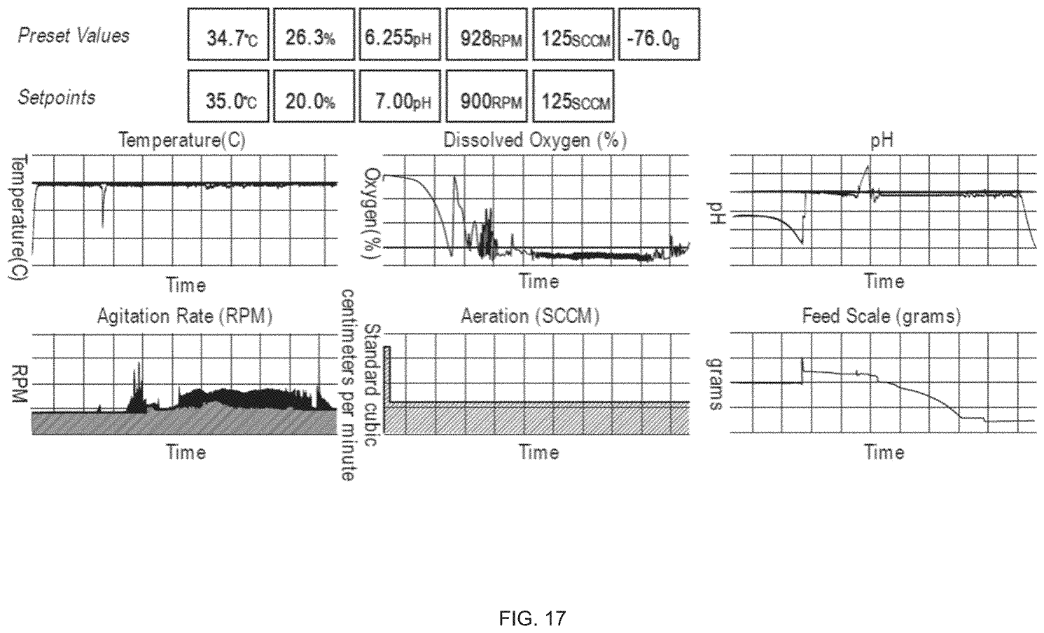

The display module can allow for online viewing and operation of the automated work cell by a user. During the experiment, the user can view the live data from the experiments on a graphical dashboard as shown in FIG. 17. A user can further see a camera feed of each experiment.

The user can query for the display of specific metrics, graphs, and imagery via a graphical user interface, a programming language, a query language or by using pre-built template dashboards. The user can compare online and offline data from experiments that are running or have run in the past. Forecasted data can optionally be displayed. The data can optionally be overlaid with information regarding the status of the protocol (e.g. what stage the protocol is in and what the controllers are trying to do). In some cases, simulated data may be displayed. Data generated at other facilities may be displayed if that data was collected previously or if the data is generated in real time from another site. In some cases, a range or “envelope” of possible variation may be displayed showing the likely bounds of a data stream.

The user can make changes to process parameters in real-time. For example, the user can modify the experimental or fermentation protocol while the fermentation system is running the pre-specified instructions. A user can also stop a fermentation run during a run, if necessary. The user may make changes without authorization from other users or staff, or the user may require further authorization. Changes in the protocol can be logged with user information and timestamps and these logs can be displayed in the display module. The user can receive a prompt or warning before finalizing a change. The user can undo some changes. The user can redo some changes. The user can see a history of the process parameters that have been changed. The user can receive a warning if they have made changes to one replica but not another. The user can see an updated protocol after the manual change has been executed.

The online dashboard as depicted in FIG. 17 can be split into two main views: first, an individual reactor view with graphs of all the live low-level sensor data of a reactor and an image feed; and second, an experimental view where higher level data from all the reactors can be plotted. After the run, the data can still be available in the dashboard and can be downloaded from the dashboard if the user chooses.

The display module can further analyze and display data from a software pipeline that processes online and offline data from a set of in vivo and simulated fermentation experiments.

The data can move from reactors to a cloud data storage where the data can first be ingested for processing. The data can then be sent from each reactor facility when a network connection is available. The data can then be cached on each reactor until a connection to the cloud computers is available. The data can be cached in an intermediate, on-premise storage device before reaching the cloud.

The data can comprise measurements, estimates, imagery, or human-readable notes. The data can come from a probe, camera, analytical device, or an end user.

The data can be entered via an API interface disclosed herein, via a web interface, via an app, from an internal system, or from an external vendor.

The ingested data can comprise a value that may be numeric or contain text in the case of a description, for example. The data can also be binary data, compressed or parseable only by certain algorithms. The data can be encrypted by an end user or by the software system.

The data can contain a timestamp that connects the value to a point in time. The timestamp can be in the past by several days or weeks in the case of an analytical assay or data uploaded from an end user. The timestamp can be in the past by several seconds in the case of a probe. The timestamp can be in the future by several seconds, minutes or days in the case of simulation and forecasting systems. Alternatively, a timespan may be used in place of a singular timestamp.

The data can have other associated metadata representing information about the data's physical location, linking the data to the physical instrument used, connecting the data to a certain protocol, connecting the data to an in vivo or simulated experiment, an indication of the confidence of the measurement, an indication of the data's provenance (e.g. in the case of a calculated metric), or an indication of the associated calibration value used to generate the metric. The calibration value can come from a calculation performed on other values. The calibration value can be down-sampled or up-sampled in the time domain. The calibration value can be marked as archived or deleted in which case the data will not be presented to end users or analyzed further.

The data can be flagged for quality control, which can trigger end users to review the accuracy of the data. Upon ingestion of the data by the cloud software, the data can be distributed into several data processing and storage pipelines.

The data can be moved to the modeling pipeline. In the modeling pipeline, software processes can aggregate the data and use certain metrics to test and refine models. The models can comprise mathematical descriptions, empirically-derived associations, equations, graph structures and parameter sets. The models can be updated on an hourly, daily, and weekly basis. Each model developed by the data can be stored for later analysis. The models can be ranked and promoted into (or demoted out of) production usage. The data can be aggregated for specific end users, specific processes, specific organisms, and collections of these subsets, including, in some cases, all subsets.

The data can be moved to the analysis pipeline. In the analysis pipeline, the analysis software can compares data to certain thresholds and other values to determine whether an alert should be raised to an end user. The analysis pipeline can generates graphs and annotates images. Other statistics can be generated including model-derived forecasts and down-sampled datasets. Some datasets can be smoothed with adjustments made according to background noise levels. Some datasets can be prepared for an end user to review; this may include resampling, frequency reduction or making the datasets sparser.

The data can be moved to the storage pipeline. In the storage pipeline, the software can store this data in a secure, access-controlled system. The data can be segregated by an end user, experiment, protocol, or organism. The data can be encrypted based on end user parameters. Other metadata can be referenced and attached to each incoming data point. The data can be temporarily added and removed from caches to facilitate fast access by other software and end-users. The data can then be sent to long term backup, including offline systems. In some cases, the data may be anonymized, removing any customer-sensitive parameters. In some cases, the data may be re-normalized to remove customer-sensitive information.

The data can be moved to the distribution pipeline. The software in the distribution pipeline can periodically package data and send the data to end users for viewing and further analysis. The data packages can comprise several channels or data from one metric representing on experiment, organism, or protocol. End users can configure the frequency of this transmission. The updated data can be sent to reactors, which can comprise updated model parameters, new metrics that are relevant for live experiments or updated forecasting results. The data can be sent to other entities including vendors for calibration checking or other personnel for annotation or quality control.

The data can be moved from one pipeline to another, and the pipelines listed in the foregoing do not have to occur in sequential order. The data generated by one pipeline can re-enter the system at the top of the ingestion funnel. The data pipelines can be hosted in one or more cloud services or they can be hosted on the end user's premises. The data processing can be configured to happen at the reactor level.

### Simulation Module

A fermentation system described herein can further comprise a simulation module. The simulation module can take the experimental protocol and simulate what could or should happen in the fermentation system with the entered experimental protocol. For new protocols, the simulation module can ensure that the user-provided protocol can run on the fermentation system. The simulation module can run live during an experimental protocol, and predict what will happen next in the steps of the experimental protocol. The simulation module can further suggest changes to the protocol to improve performance.

Additionally, the simulation module can highlight when the live conditions have diverged from expected performance. The simulation module can be improved with additional run data. The simulation module can be adjusted for each type of fermentation agent and substrate. The performance can be updated as changes are made to the protocol (e.g. to the fermentation agent, the fermentable substrate, or to the protocol steps). The simulation module can run on a computing system at each bioreactor or on a user's laptop or on a plurality of machines on premise or off-premise.

The simulation module can also run live during an experiment, and alter the protocol in real-time to improve performance. An experiment protocol can be supplied by the end user. If another user makes a suggestion to modify the protocol, then that adjusted value can be partially simulated or simulated from that point onward.

The simulation module can alert the user that a proposed experimental protocol is incompatible with the present system. The simulation module can further predict, for example, performance, efficiency, yield, and output of the experimental protocol.

The simulation module can simulate the end-result that a user of the system is trying to achieve. For example, a user can be trying to achieve a specific biomass of a fermentation product or fermentation agent, or a particular product.

The simulation module can validate whether an input protocol is valid before the experimental protocol is run. The input experimental protocol can be evaluated for feasibility given a certain set of hardware, sampling requirements, or liquid additions. For instance, if in an experimental protocol, there is a step requiring addition of a liquid to a vessel in a system disclosed herein, and if the liquid additions would cause the vessel to overflow, then the simulation module can issue a warning to the user. In another case, if the sampling requirements would remove too much liquid from the vessel, then the simulation module can issue a warning to a user or mark the experimental protocol as invalid.

The input experimental protocol can be evaluated in terms of incompatible control strategies. For example, if the experimental protocol specifies a constant feeding profile along with a constant mixing and aeration strategy, then the simulation module can warn a user that the oxygen values will not be sufficiently controlled.

Further, if the experimental protocol is designed to trigger a certain phase with the pH increases, but if the experimental protocol uses acid control to prevent this increase, then the simulation module can issue a warning to the user or mark the experimental protocol as invalid.

A simulation can be paused at any point. The simulation module can capture the state of a running simulation and restart a simulation from that point to save time or computation.

The simulation module can utilize Monte Carlo techniques to predict performance in noisy environments. The simulation module can further be run in an idealized mode where actuators perform exactly to the user-provided specifications, and the modeled biological systems behave exactly as intended.

The simulation module can also be run in a more probabilistic mode, where actuators and biological parameters react with a distribution of outcomes, governed by a distribution choice and associated parameters. The distribution can be, for example, Gaussian, with a specified mean and variance. For example, when a cell reacts to a change in nutrient conditions, the cell may grow, die, metabolize, undergo a metabolic shift, or signal to neighboring cells at rates and probabilities governed by a distribution. The simulation module can take these distributions as an input and, in a high throughput fashion, be distributed to run across multiple processes, threads, cores or machines. The simulation module, running in parallel, can forecast an experimental protocol and predict certain parameters at given time points. The predicted parameters can be, for example, the product titer, specific growth rates, metabolite concentrations, cell viability, or off gas evolution rates. Each simulation module can contribute its estimate of the final value, given the input model, experimental protocol, input conditions and distribution of the inputted parameters. These output values can fall on a certain distribution that the simulation module can analyze and coalesce into one value: an expected value and a confidence level.

For example, the final product titer can be predicted with some confidence, given an array of many possible experimental conditions. The simulation module can run in this mode with respect to experimental conditions or experimental protocol parameters that fall on a distribution.

The biological model used for simulation, and the model governing the biological reactivity of the system can be controlled by the end user. The end user can further specify a set of parameters that govern reactions to metabolites. These parameters can describe primary, secondary, and tertiary metabolic pathways that are present in the organism. The simulation module can use these values to control the simulated organism and the organism's response to nutrient levels and experimental protocol conditions.

During a live experiment with a running experimental protocol, the simulation module can forecast the outcome at varying degrees of precision. When forecasting over the next few seconds, the simulation module can make an accurate prediction of the state of the system: the experimental protocol parameters (e.g. broth temperature, pH, oxygen levels) and the state of the actuators (e.g. mixing rates, pumping rates). The simulation module can feed these predictions to controllers on different channels. The resulting data can be utilized to adjust actuator state based on predicted future changes. End users can also view this data and decide whether an experiment should continue operating. Some experiments can be forecasted to perform poorly over the long term and an end user can decide to terminate the experiment before a scheduled end time for the experiment. The forecasts can come with some estimate of confidence: forecasts made over longer timespans can have reduced confidence. End users and internal software can make use of the confidence data as well as the forecasted parameters.

Simulation experiments can be started via a web browser, mobile device, desktop, or mobile application interface. The end user can define, run, and analyze simulations as though they were any other experiment in the system. The simulation data can be compared to other in vivo experiments or other simulation experiments, or any combination of the foregoing.

Simulation results can be viewed by teams and discussed in a web-based interface. The planning of future simulations can be coordinated via the web tool. Simulations can be defined and then tested on a small set of computing resources (e.g. cores, processes, devices) and then the same test can be performed on a larger number of devices to improve throughput, simulation fidelity, or statistical accuracy.

Simulation data can be analyzed in the web-based tool or downloaded for analysis by other tools. The analysis can comprise viewing metrics, statistics, and graphs to compare simulation data with other in vivo and simulated experiments.

### Comparison Module

A fermentation system described herein can further comprise a comparison module. The comparison module can compare data from multiple bioreactors in a single batch. The batch can comprise one or more different experimental protocols being run simultaneously. The comparison module can further compare data from multiple batches. The comparison module can further compare past and present data, and data provided by the simulation module. The comparison module can compare data generated by different equipment and/or at different facilities.

The comparison module can calculate statistics to quantify the variability between experiments. For example, the comparison module could calculate the variation between two or multiple experiments with the same strain, media, and protocol, to determine the reproducibility of the experimental results (which could be the growth rates, product titers, or any other process parameter).

The comparison module can be used to identify variation between individual components of the system, for example, the heating and cooling system, and calculate statistics comparing the operation of these systems.

Additionally, the comparison module can delineate experiments that perform above a certain threshold. The comparison module can find experiments that experienced a shared set of characteristics or control strategies or similarities in their protocols. The comparison module can take multiple experimental parameters into account and determine which parameter has the largest impact on performance. The comparison module can show these comparisons, for example, via graphs, statistics, image-overlays, tables, or charts.

The system may include any other modules in addition to the aforementioned components. For example, the system may further include an Internal Review module that allows customers and management users to analyze the efficacy of the system including but not limited to, “how many runs are performed with which strain”, “what was the utilization of reactors and other analytical equipment”, “what is the inventory status now and projected out into the future, given certain experimental plans, “what is the projected team workload, given certain experimental plans,” “how many runs were performed on certain strains and how accurately are the processes executed for each strain type”, etc.

### Input and Users for the Fermentation System

The input of the system can comprise, for example, an experimental protocol, a fermentation protocol, a duration for the fermentation process, a fermentation agent or microbial strain to be used in a system disclosed herein, a fermentable substrate or culture medium to be used in a system disclosed herein, the pH of the culture medium, the number of work cells or bioreactors that can be used during the fermentation process, or the temperature at which the fermentation process should be run.

A user of a system described herein can be, for example, a scientist, researcher, manufacturers, or a biologist.

A user of the system described herein can access a fermentation system described herein from, for example, a computer system. The user can enter this information into an input module of the system and the system can use this information to create a fermentation protocol. The user can further enter information via a cloud-based access system, wherein the cloud-based access system can be connected to a computer system disclosed herein. The user can post, for example, comments and notations on any user-provided protocol, or a protocol that is part of a database of a system described herein.

The output of a system disclosed herein can be displayed, for example, as a webpage, web-based application, a module, a dashboard, or a graphical interface. The system can be a software application that can be installed on, for example, a computer, a cell phone, a laptop, or a tablet.

Any tool, interface, engine, application, program, service, command, or other executable item can be provided as a module encoded on a computer-readable medium in computer executable code. In some embodiments, the present disclosure provides a computer-readable medium encoded therein computer-executable code that encodes a method for performing any action described herein, wherein the method comprises providing a system comprising any number of modules described herein, each module performing any function described herein to provide a result, such as an output, to a user.

### Fermentation

Fermentation processes can be used for many applications. For instance, fermentation can be utilized for production of biomass (e.g., viable cellular material), production of extracellular metabolites (chemical compounds), production of intracellular components (e.g., enzymes and other proteins), or transformation of a substrate (e.g., the substrate itself can be a product). Fermentation processes are useful for biological experiments, drug manufacturing, food industry, biofuels, or many other applications. In some instances, it can be desirable to provide automated fermentation systems and methods that allow for low risk of contamination, high levels of accuracy and repeatability, high throughput, controlled variations, quicker turnaround, and/or require less manpower.

Fermentation can use a fermentation agent to transform a fermentable substrate (which can include, for example, Carbon, Oxygen, Nitrogen, Hydrogen, Sulfur, Calcium, and a number of trace elements) into some useful product. Fermentation can be an aerobic process (i.e. one that uses oxygen) or anaerobic (a process that occurs in oxygen limited or restricted environments). A fermentation agent can be, for example, yeast, bacteria, algae, fungi, mammalian cells, animal cells, and insect cells, a microbial strain, or any combination thereof

Non-limiting examples of a yeast that can be used in a system described herein include Saccharomyces cerevisiae, S. oviformis, S. chevalieri Schizosaccharomyces pombe, S. uvarum, S. carlsbergensis, Sch.malidevorans, Kluyveromyces, Debaromyces, Hanseniaspora, Issatchenkia, Pichia, Candida, S. bisporus, S. rouxii, Zygosaccharomyces; S. rosei, Torulaspora; and T delbrueckii.

Non-limiting examples of a bacterium that can be used in a system described herein include Streptococcus, Lactobacillus, Bacillus, Lactococcus, Lactococcus, Propionibacterium, Escherichia, Enterobacter, Clostridium, Streptococcus lactis, S. cremoris, Leuconostoc mesenteroides, L. dextranicum, L. lactis, L. cremoris, L. paramesenteroides, L. oenos, Pediococcus cerevisiae, P. acidilactici, P. pentosaceus, P. halophilus, Enterococcus, Lacotbacillus, delbrueckii, L. leichmannii, L. lactis, L. bulgaricus, L. helviticus, L. acidophilus, L. casei, L. plantarum, Leuconostoc, Lacotbacillus fermentarum, L. brevis, L. buchneri; may produce manitol and dextran; Lactobacillus, Pediococcus, Leuconostoc, Clostridium butyricum (1,2); Cl. Acetobytylicum (1,3,4,5); Cl. kluyveri, Cl. aceticum (1, 6); Propionigenium modestum, Oxalobacterformigenes; Anaeroplasma and Arthrobacte, and Acetobacterium.

Non-limiting examples of a fungus that can be used in a system described herein include Mucor (e.g., M. miehei), Rhizopus (e.g., R. oligosporus), Alternaria, Aspergillus, A. rugulosus, Botrytis (e.g., B. cinerea), Cladosporium, Colletotrichum, Fusarium, Monilia, Penicillium, Trichothecium, Aureobasidium (Pullularia), Geotrichum, and G. candidum.

A fermentation product can be, for example, acetic acid, citric acid, ethanol, antibiotics, proteins, enzymes, monoclonal antibodies, the organisms themselves, cheese, wine, or beer.

A fermentable substrate (e.g., a carbon source) can comprise, for example, a polysaccharide, a monosaccharide, a carbohydrate, a sugar, a starch, methane, carbon dioxide, carbon monoxide, or cellulose. In some embodiments, culture media includes complex fermentable substrates containing a variety of carbon sources, such as yeast extract. Media also can include salts, nitrogen sources, trace metal elements, and other components for growing biomass and producing fermentation products.

A fermentation system described herein can contain a work cell that can be automated and programmable and controlled by a cloud interface. A user of the system can design an experiment on, for example, a web interface that can automatically be checked by a software system described herein. An experiment designed by the user can be composed of, for example, a fermentation agent, such as a microbial strain, a fermentable substrate, such as a culture medium for the fermentation agent, and a fermentation protocol.

The user can define the fermentable substrate and fermentation agent to be used in a fermentation protocol. The user can select pre-defined protocols that are part of a database of the system, or the user can customize a protocol. The software system of the present disclosure can check for any configurations needed in the hardware based on the proposed experimental design. The user can view the progress of the fermentation protocol when the work cell is open and then schedule runs. The software of the present system can view the desired experiment and the existing demand, and then calculate a cost/bioreactor-run for the user. The experimental protocol and schedule provided by the user can be turned into a set of instructions for the fermentation system. The present software can turn the user-defined experimental protocol into an operator-robotic program.

During a run, data can come into the cloud system from many sources in and out of the work cell including, for example, data from a bioreactor within the work cell, data from pieces of analytical equipment that can be automatically run inside the work cell, data from software or computer vision-based sensors that can track the movement of operators coming in and out of the work cell, and temperature sensors that can monitor the ambient condition of the work cell or the surrounding environment.

During a run, a user can view the live data from, for example, the work cell and images from the experiment, and then make real-time changes to the protocol parameters, which can include changing process set points or swapping in an entirely new protocol. Additionally, the user can stop or pause the run based on the data feed. Based on the data coming into the system, the cloud system of the present disclosure can make live estimations of important parameters during the experiment. After a run, the user can view the data from the experiment in the protocol. An API can also allow a user to design a protocol, schedule an experiment, ingest live data, and control the experiment.

While various embodiments of the invention have been shown, and described herein, it will be obvious to those skilled in the art that such embodiments are provided by way of example only. Numerous variations, changes, and substitutions can occur to those skilled in the art without departing from the invention. It should be understood that various alternatives to the embodiments of the invention described herein can be employed.

### EXAMPLES

### Example 1: Computer Control Systems

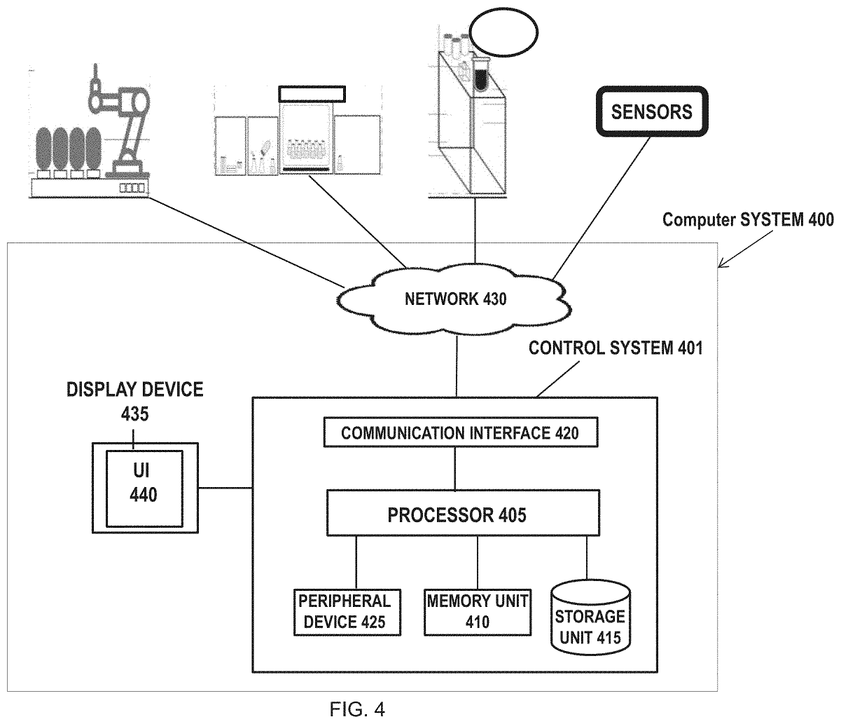

The present disclosure provides computer control systems that are programmed to implement methods of controlling a fermentation system as described herein. FIG. 4 shows a computer system 401 that is programmed or otherwise configured to control a fermentation system based on user input of an experimental and fermentation protocol.

The computer system can be an electronic device of a user or a computer system that is remotely located with respect to the electronic device and a user interface 440. The electronic device can be a mobile electronic device.

The computer system 400 can include a control unit 401 and central processing unit 405 (CPU, also “processor” and “computer processor” herein), which can be a single core or multi core processor 405, or a plurality of processors for parallel processing. The computer system 400 can also include a memory unit or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU through a communication bus, such as a motherboard. The storage unit 415 can be a data storage unit (or data repository) for storing data. The computer system 400 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 400, can implement a peer-to-peer network, which can enable devices coupled to the computer system 400 to behave as a client or a server. The communication network(s)can include local area networks (LAN) or wide area networks (WAN), such as the Internet. The communication network(s)can comprise telecommunication network(s) including transmitters, receivers, and various communication channels (e.g., routers) for routing messages in-between. The communication network(s)can be implemented using any known network protocol, including various wired or wireless protocols, such as Ethernet, Universal Serial Bus (USB), FIREWIRE, Global System for Mobile Communications (GSM), Enhanced Data GSM Environment (EDGE), code division multiple access (CDMA), time division multiple access (TDMA), Bluetooth, Wi-Fi, voice over Internet Protocol (VoIP), Wi-MAX, or any other suitable communication protocols.

The control system 401 or CPU 405 can execute a sequence of machine-readable instructions, which can be embodied in a program or software. The instructions can be stored in a memory location, such as the memory 410. The instructions can be directed to the control system or CPU 405, which can subsequently program or otherwise configure the control system 401 or CPU 405 to implement methods of the present disclosure, including experimental and fermentation protocols provided by a user. Examples of operations performed by the control system or CPU 405 can include fetch, decode, execute, and writeback.

The CPU 405 can be part of a circuit, such as an integrated circuit. One or more other components of the system 400 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 400 in some cases can include one or more additional data storage units that are external to the computer system 400, such as located on a remote server that is in communication with the computer system 400 through an intranet or the Internet.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 400, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type medium can include any or all of the tangible memory of the computers, processors or the like, or associated modules thereof, such as various semiconductor memories, tape drives, disk drives and the like, which can provide non-transitory storage at any time for the software programming. All or portions of the software can at times be communicated through the Internet or various other telecommunication networks. Such communications, for example, can enable loading of the software from one computer or processor into another, for example, from a management server or host computer into the computer platform of an application server. Thus, another type of medium that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as medium bearing the software. As used herein, unless restricted to non-transitory, tangible “storage” medium, terms such as computer or machine “readable medium” refer to any medium that participates in providing instructions to a processor for execution.

Hence, a machine readable medium, such as computer-executable code, can take many forms, including but not limited to, a tangible storage medium, a carrier wave medium or physical transmission medium. Non-volatile storage medium include, for example, optical or magnetic disks, such as any of the storage devices in any computer(s) or the like, such as can be used to implement the databases, etc. shown in the drawings. Volatile storage medium include dynamic memory, such as main memory of such a computer platform. Tangible transmission medium include coaxial cables; copper wire and fiber optics, including the wires that comprise a bus within a computer system. Carrier-wave transmission medium can take the form of electric or electromagnetic signals, or acoustic or light waves such as those generated during radio frequency (RF) and infrared (IR) data communications. Common forms of computer-readable medium therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable medium can be involved in carrying one or more sequences of one or more instructions to a processor for execution.

The computer system 400 can include or be in communication with an electronic display 435 that comprises a user interface (UI) 440 for providing, for example, the displays depicted in any of the other figures including, for example, visualization of data during a fermentation run, set-up of a proposed fermentation protocol, the status of a fermentation run, and selection of a fermentation agent and fermentable substrate. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, implement various methods of machine learning to generate predictive models and forecasts that can be used to predict, for example, the status and result of fermentation processes run on a system disclosed herein.

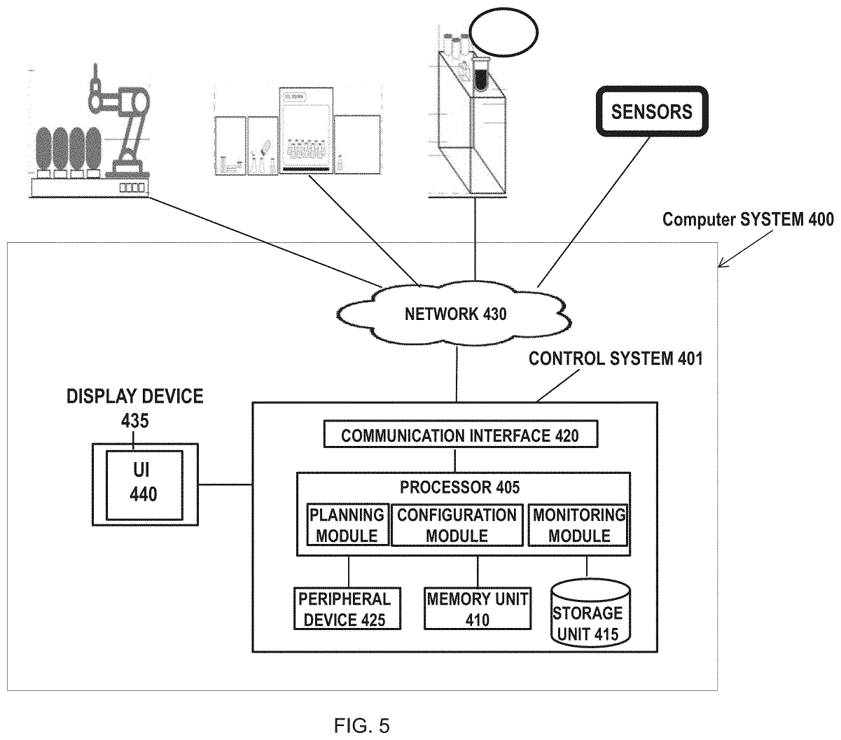

FIG. 5 depicts the computer system of FIG. 4; however, FIG. 5 shows that that the processor 405 can comprise a planning module, configuration module, and monitoring module.

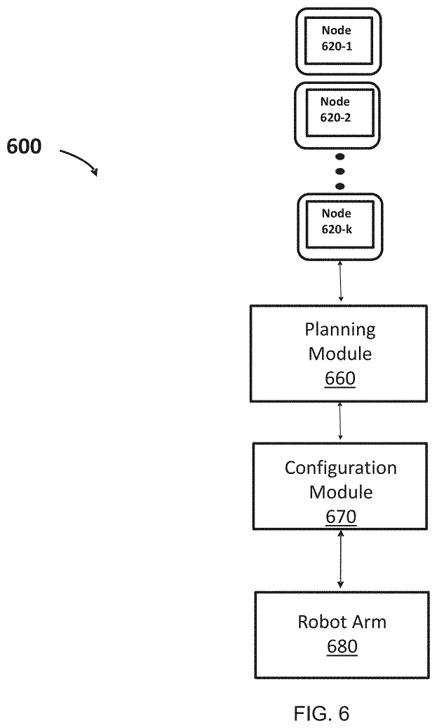

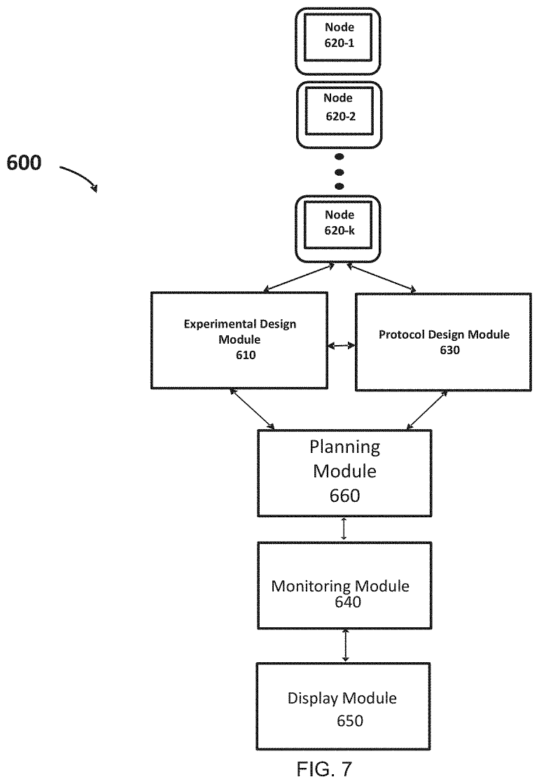

FIGS. 6 and 7 shows an exemplary network 600 used by a system described herein. The network 600 can comprise a plurality of nodes 620-1, 620-2, and 620-k. A node can be any device equipped with communication capabilities, which communication capabilities can be cloud-based. The communications can be wired or wireless communications. The node can be operating over various technologies such as Global System for Mobile communication (GSM), Universal Mobile Telecommunications System (UMTS), High Speed Downlink Packet Access (HSDPA), Code Division Multiple Access (CDMA), Long Term Evolution (LTE), Worldwide Interoperability for Microwave Access (WiMAX) and infrastructure IEEE 802.11 variants, such as IEEE 802.11a/b/g/n/ac, or a cloud-based system, and various others. A node can be a Bluetooth or Wi-Fi enabled device, such as laptops, cellular phones, Personal Digital Assistant (PDA), Smartphone, HSDPA terminal, CSMA terminal and various other access terminals. A node can operate as a broadcast node, relay node, source node, sink node or recipient node in the network. A node can or cannot be mobile, or cloud-based.

A node can be various types of computing devices such as personal computers, tablet computers, smart phones, set top boxes, desktop computers, laptops, gaming systems, servers, data centers, and various other devices or systems. A node can be any type of network devices. The plurality of nodes can establish communications with others devices or systems in the network (e.g., experimental design module 610, protocol design module 630, monitoring module 640, display module 650, planning module 660, configuration module 670, other third party server, etc). The network can be a wireless network, wired network, a cloud-based network, or any combination thereof. For example, the network can comprise one or more of the Internet, an intranet, a cellular network, a home network, a person area network, etc., through an ISP, cellular, or broadband cable provider, and the like. The network can comprise an internet protocol interfaces, such as one or more network components, data servers, connection nodes, switches, and the like. In some cases, the plurality of nodes can be considered as part of the network. The plurality of nodes can be configured to run any suitable applications for conducting a transaction.

The network can comprise an experimental design module 610 for a user to provide a desired experimental protocol, which experimental protocol can contain one or multiple bioreactor runs. Each bioreactor run can be composed of a strain being tested, the set of medium being used, and the protocol being used. In some embodiments, the experimental design module can be implemented on a server. The experimental design module can optionally be implemented on a network device. The experimental design module can be implemented by software, hardware or a combination of both.

The experimental design module 610 can be in communication with the protocol design module 630 over the network, which network can be cloud-based. The protocol design module 630 can be configured to receive the experimental protocol from the experimental design module. The protocol design module can contain the strains to be used, the culture medium that will be used, a series of set-points that can be constant through a fermentation run or a time variable, and information to control the bioreactor pumps and motors based on sensors attached to the fermentation system. The sensors can include sensors for pH, dissolved oxygen, and temperature. Other software sensors can include reactor volume, cell concentration, cell metabolism, and an estimate of the carbon substrate in the reactor. The protocol can also include a set of steps for the seed train, medium preparation, sample preparation, and storage. In some embodiments, the protocol design module is implemented on a server. The protocol design module can optionally be implemented on a network device. The protocol design module can be implemented by software, hardware or a combination of both.

The experimental design module 610 and the protocol design module 630 can be in communication with the monitoring module 640. The monitoring module 640 can assess the suitability of the experimental protocol provided by the experimental design module 610 and the fermentation protocol provided by the protocol design module 630 for the fermentation system. The monitoring module can optionally be implemented on a network device. The monitoring module can be implemented by software, hardware or a combination of both.

The experimental design module 610, the protocol design module 630, and the monitoring module 640 can be in communication with the display module 650. The display module 650 can display live data as a fermentation run proceeds. A user can make changes to the experimental or fermentation protocol in real-time as the fermentation run progresses. The display module can show an individual reactor view and an experimental view.

A portion of the example network 600 is shown in FIG. 6. FIG. 6 shows that via the nodes connected to the planning module 660, a user can enter information regarding, for example, the fermentation system protocol. The information entered by the user in the nodes 620-1, 620-2, 620-k, and so on, is relayed to the planning module. The information entered into the planning module can then be relayed to the configuration module 670, which can provide instructions regarding, for example, movement, to a robot arm 680.

A portion of the example network 600 is also shown in FIG. 7. FIG. 7 shows that the information entered by the user in the nodes 620-1, 620-2, 620-k, and so on, can be relayed to the experimental design module 610 and the protocol design module 630. The experimental design module 610 and the protocol design module 630 can then relay that information to the planning module 660, and as shown in FIG. 6, the planning module 660 can send that information to the configuration module 670 and then to the robot arm 680. Any information entered into, for example, the experimental design module 610, protocol design module 630, planning module 660, and configuration module 670, can be monitored by the monitoring module 640 to determine the suitability of any provided instructions for the fermentation system described herein. The experimental design module 610, the protocol design module 630, planning module 660, and the monitoring module 640 can be in communication with the display module 650. The display module 650 can display live data as a fermentation run proceeds. A user can make changes to the experimental or fermentation protocol in real-time as the fermentation run progresses. The display module can show an individual reactor view and an experimental view.

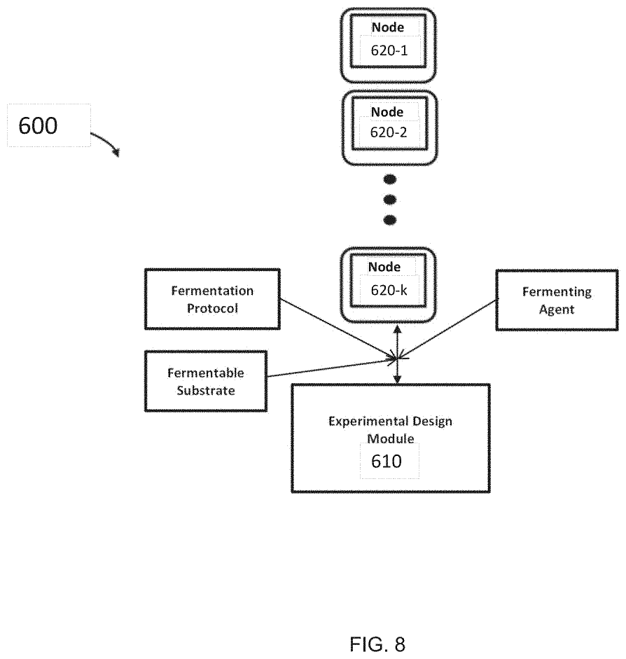

A portion of the example network 600 is shown in FIG. 8. FIG. 8 provides a zoomed-in view of the experimental design module 610, and shows that via the nodes connected to the experimental design module 610, a user can enter information regarding, for example, a fermentation agent, a fermentable substrate, and a fermentation protocol. The information entered by the user in the nodes 620-1, 620-2, 620-k, and so on, is then relayed to the experimental design module.

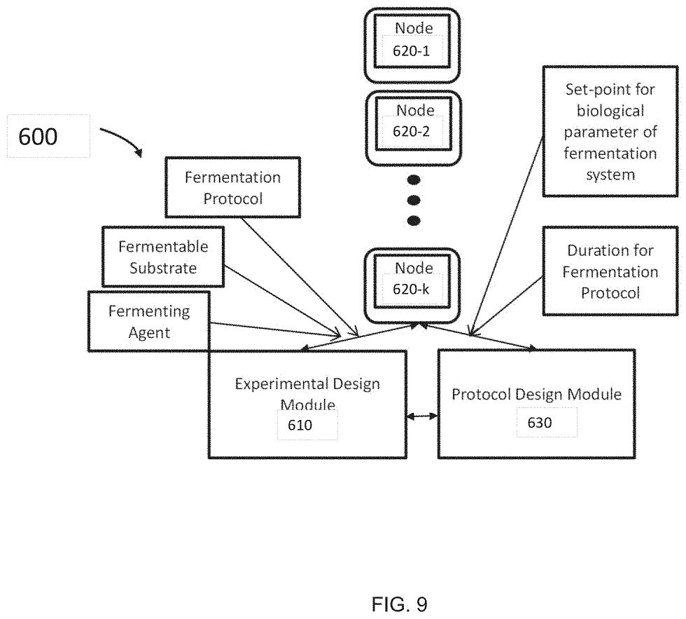

FIG. 9 shows a portion of the example network 600 containing the experimental design module 610 and the protocol design module 630. FIG. 9 provides a zoomed-in view of the experimental design module 610 and the protocol design module 630, and shows that a user can enter in information regarding a duration for the fermentation protocol received from the experimental design module 610, and a set-point for a biological parameter of the fermentation system. Additionally, the protocol design module can 630 receive information entered via the nodes to the experimental design module 610 by the user.

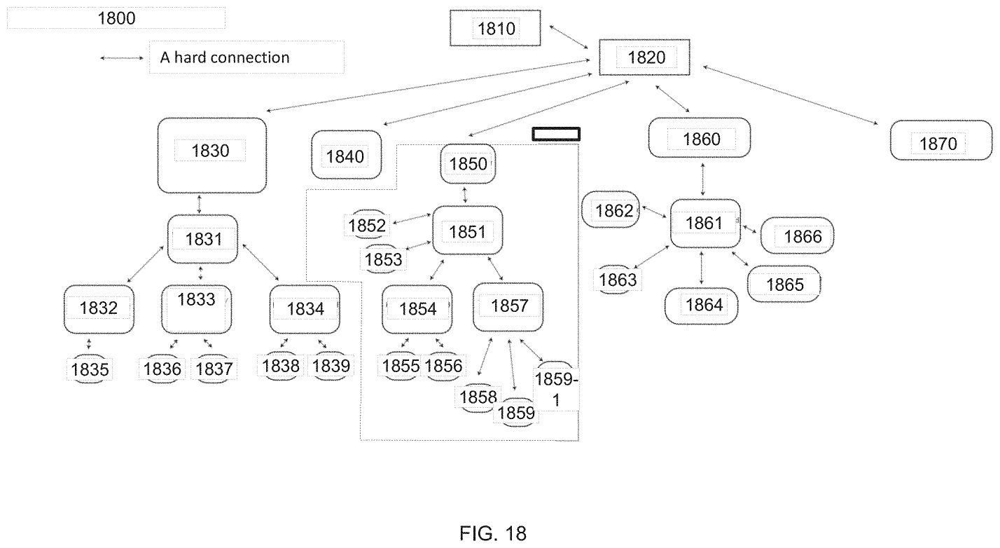

FIG. 18 shows an example of an electronics/communications architecture 1800, in accordance with embodiments described herein. Such architecture is provided by way of example only, and is not limiting. Alternative components or modules can be utilized.

A cloud server 1810 can communicate with a work cell over a network 1820. Any description herein of a cloud server can apply to one or more server computers. In some instances, a cloud computing infrastructure can be utilized. Optionally, peer-to-peer infrastructure can be utilized. A cloud server can be remote to a work cell. The cloud server can optionally be at a different room, building, address, city, state, or country from the work cell, or cannot be required to be at any of these locations. The cloud server can be in the same room, building, address, city, state, or country as the work cell.

The cloud server can include or communicate with a remote terminal through which a user can interact with the system. A user can optionally be an individual running one or more experiments in the work cell, or managing the work cell. Optionally, multiple remote terminals and/or users can have access to the system. A remote terminal can optionally be outside a work cell. A remote terminal can be at a different room, building, address, city, state, or country from the work cell.

The cloud server can communicate with the work cell over a network 1820. Any type of network can be employed. For instance, a local area network (LAN), or wide area network (WAN), such as the Internet can be employed. The network can be a telecommunications network.

The work cell can comprise a work cell computer 1830. The work cell computer can be physically within the work cell, or can be outside but directly communicating with components of the work cell. The work cell computer can communicate with a work cell embedded system 1831. A work cell embedded system can communicate with an environmental sensor embedded system 1832 (which can optionally comprise one or more sensors 1835, such as sensors used in environmental monitoring), a carriage pumping/scales embedded system 1833 (which can optionally comprise scales 1836, such as scales, and/or pumps 1837), and/or a linear rail embedded system 1834 (which can comprise one or more motors 1838 and/or sensors 1839, of a linear rail 1852).

The work cell computer can or cannot have user interface that can allow a user to directly interact with the work cell computer. Communications between the work cell computer and the various systems can be hardwired, or can be wireless. The work cell computer can communicate with the cloud server over a network.

The work cell can comprise a robot 1840. The robot can have any characteristics or features as described elsewhere herein. The robot can communicate with a cloud server over a network. The robot can communicate information about a robot status, robot location, robot position, or robot activity. In some instances, a robot can receive instructions from a cloud computer, work cell computer, or any other device as described elsewhere herein. For example, a robot can be instructed to perform a particular task. A robot can be capable of performing steps autonomously or semi-autonomously. In some instances, a robot can be provided with a task, and the robot can be able to execute the task within the environment without detailed direction and based on information collected by sensors (e.g., on-board and/or off-board the robot). A robot can have one or more processors on-board the robot that can be capable of generating instructions for execution by the robot. The robot can have one or more processors that can execute code, logic or instructions for performing one or more steps. The robot can have one or more memory storage unit that can comprise non-transitory computer readable media comprising code, logic, or instructions for performing one or more steps.

In some embodiments, a bay computer 1850 can be provided in a work cell. A bay computer can be provided for each bay (e.g., bioreactor). The bay computer can communicate with a bay embedded system 1851, which can communicate with one or more sensors 1852 and/or motors 1853 for the bay. The bay embedded system can optionally communicate with a pumping embedded system 1854, which can comprise one or more pumps 1855 and/or scales 1856. The bay embedded system can communicate with a thermal embedded system 1857 which can comprise sensors 1858, pumps 1859, and/or heaters/coolers 1859-1. Such systems can be provided for each bay. Such systems can be capable of operating independently of systems in other bays.

The bay computer can or cannot have user interface that can allow a user to directly interact with the bay computer. Communications between the bay computer and the various systems can be hardwired, or can be wireless. The bay computer can communicate with the cloud server over a network. The bay computer can optionally communicate with a work cell computer, and/or a robot directly or over a network.

A work cell can comprise a seed train computer 1860. The seed train computer can interact with a seed train embedded system 1861. The seed train embedded system can comprise a bar code reader 1862, scales 1863, incubator 1864, incubator environmental sensors 1865, and/or live optical density sensing 1866. Any other sensors or equipment described elsewhere herein, including those related to the seed train station, can be included herein.

The seed train computer can or cannot have user interface that can allow a user to directly interact with the seed train computer. Communications between the seed train computer and the various systems can be hardwired, or can be wireless. The seed train computer can communicate with the cloud server over a network. The seed train computer can optionally communicate with a work cell computer, a robot, and/or a bay computer directly or over a network.

A sample analysis computer 1870 can be provided, optionally within a work cell. The sample analysis computer can optionally interface with any systems or equipment within the work cell. The sample analysis computer can interface with any sample handling sensors, equipment or other components described elsewhere herein.

The sample analysis computer can or cannot have user interface that can allow a user to directly interact with the sample analysis computer. Communications between the sample analysis computer and the various systems can be hardwired, or can be wireless. The sample analysis computer can communicate with the cloud server over a network. The sample analysis computer can optionally communicate with a work cell computer, a robot, a bay computer, and/or a seed train computer directly or over a network.

The systems and methods provided herein can allow for an automated fermentation work cell to perform experiments in a fully automated fashion. This can allow for communication between the various components to perform the fermentation processes, which can include the seed train preparation, the fermentation, sample preparation, and/or sample analysis. The systems and methods provided herein also allow the work cell to be modular, which can permit flexibility in running various experiments.

Any embodiment of the invention described herein can be, for example, produced and transmitted by a user within the same geographical location. A product of the invention can be, for example, produced and/or transmitted from a geographic location in one country and a user of the invention can be present in a different country. In some embodiments, the data accessed by a system of the invention is a computer program product that can be transmitted from one of a plurality of geographic locations to a user. Data generated by a computer program product of the invention can be transmitted back and forth among a plurality of geographic locations, for example, by a network, a secure network, an insecure network, an internet, or an intranet. In some embodiments, a system herein is encoded on a physical and tangible product.

### Example 2

### Use of a System Disclosed Herein to Develop a Fermentation Protocol for the Fermentation System

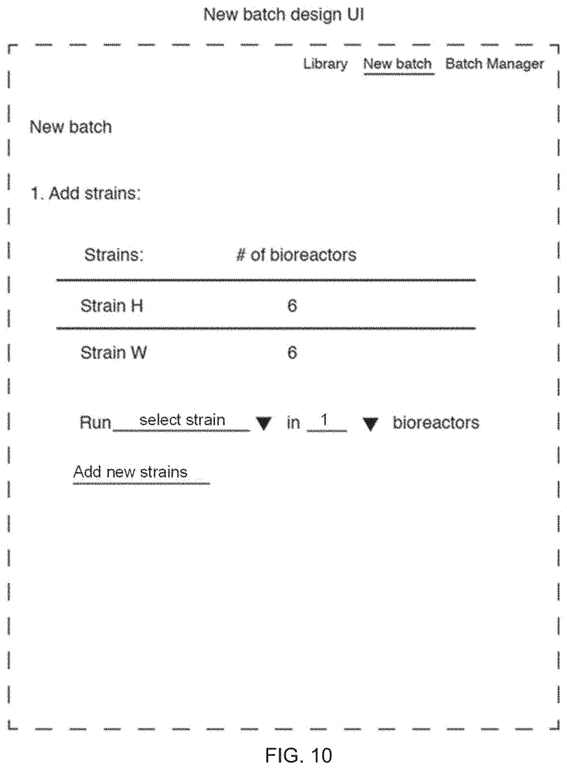

FIG. 10 provides an illustrative example of a user interface displayed to a user upon entry into the system website or API. At the initial display, the user enters different experiments to be run, along with the particular microbial strain to be used during the fermentation process. Under the process tab, the user enters in processes, or experimental protocols, to be run. The experimental protocol can be custom to the user, or be loaded by the system from a database of experimental protocol stored on the system's server.

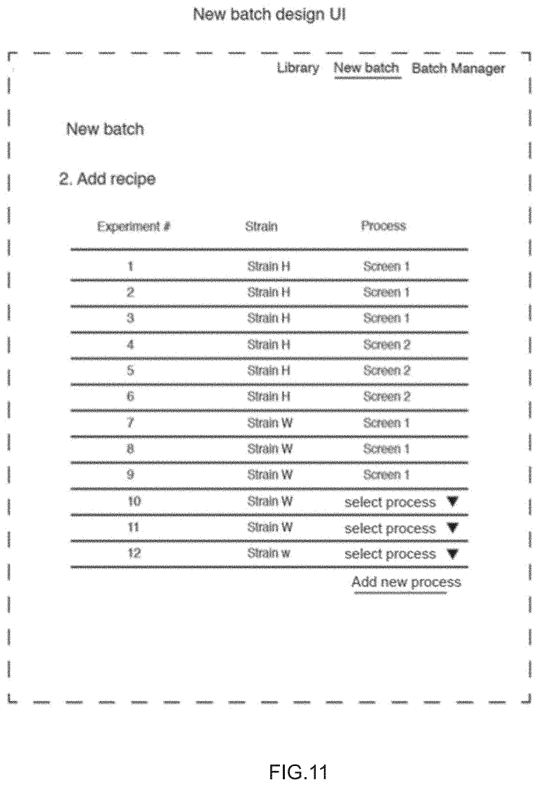

FIG. 11 provides more details regarding the planned experimental protocol provided by the user. The right columns of the FIG. 11 shows selection of a microbial strain and a process to be used in a user-selected experiment. The columns can automatically populate based upon the selected experimental process. The culture medium selected by the user can be culture medium provided by the user.

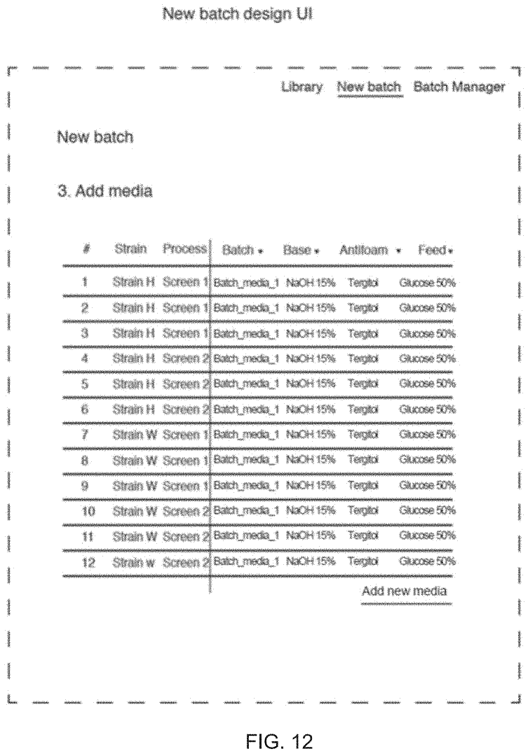

FIG. 12 provides a general overview of how the fermentation process will run based upon the experimental protocol entered by the user. The top portion of FIG. 12 indicates the type of medium, the name of the protocol, the base, feed, and anti-foam properties of the culture medium, the microbial strains to be used, and the number of bioreactors that will be used during the fermentation process.

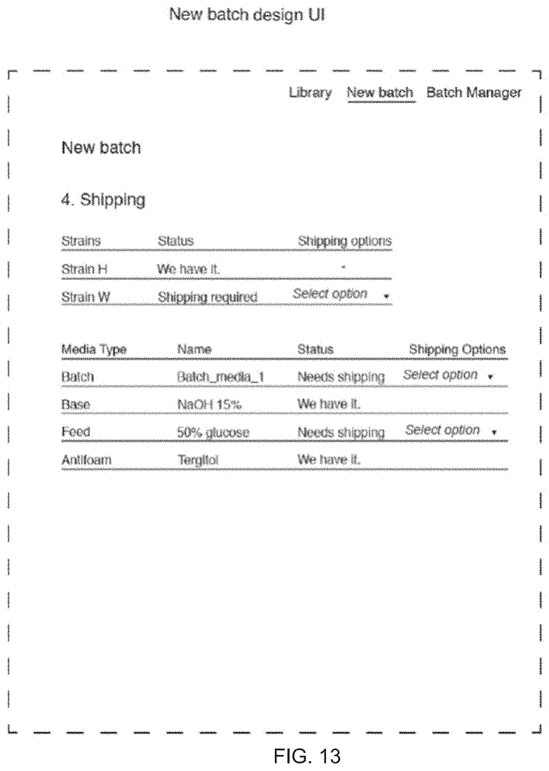

FIG. 13 shows that when a user enters in particular parameter for a fermentation process, the system can determine the status of that parameter, for example, whether the microbial strain or the culture medium must be shipped or is ready for use.

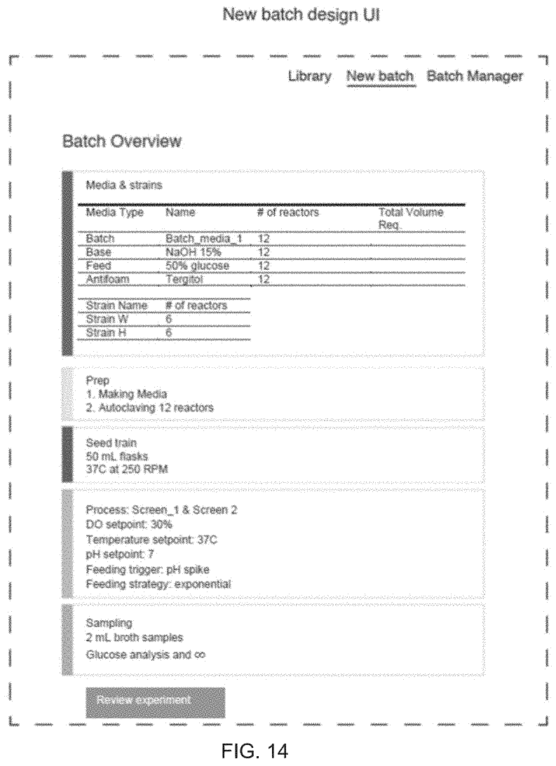

FIG. 14 shows how the different microbial strains selected by the user can be distributed over the number of bioreactors selected by the user. The bottom portion of FIG. 14 describes the steps that will be taken to proceed with the fermentation process. First, the culture medium is prepared, and the bioreactors are autoclaved. Second, 50 mL flasks containing the medium and the microbial strain are placed at 37° C. and shaken at 250 rpm. The process panel indicates that the dissolved oxygen (DO) is set at 30%, the temperature is set at 37° C., feeding is triggered when there is a pH spike, and the feeding is done at an exponential rate. Finally, toward the end of the fermentation process, 2 mL of the culture medium is sampled for a glucose and optical density (OD) analysis.

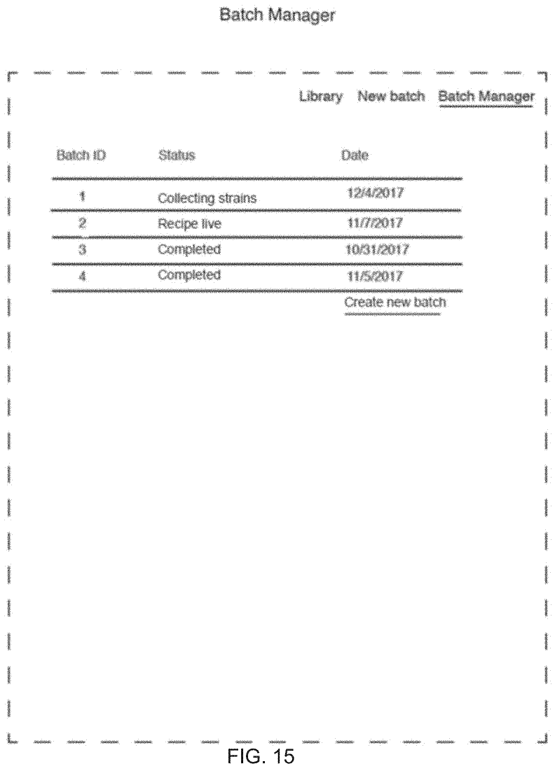

FIG. 15 provides the status of different fermentations run by the user (batches). Each status is an active link. For example, when the status states “collecting strains,” the user can click on the link and be taken to an overview of the scheduled protocol. When the status states “recipe live,” the user can click on the link and view live data. When the status states “complete,” the user can click the link and be taken to graphs and data from the previously run experiment.

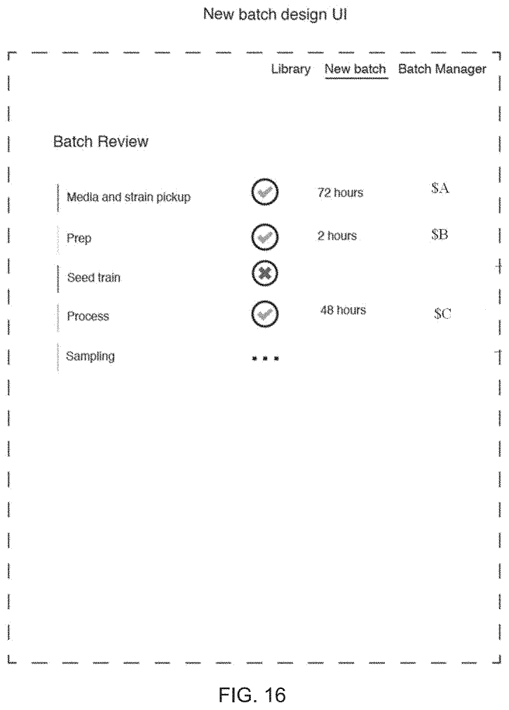

FIG. 16 shows a review of an experimental protocol selected by the user. A checkmark indicates that the specific step of the process passed review. An x-mark indicates that the specific step failed the check and requires user attention. The right-most column provides cost estimates (A, B, C) for each step of the fermentation process, allowing a user to manage cost constraints.

FIG. 17 shows a dashboard that a user sees as a fermentation run proceeds. The dashboard provides data relating to, for example, temperature, dissolved oxygen, pH, spin rate (rpm), aeration (SCCM), and feed scale (grams). The dashboard provides the set-points, actual values, and graphical representations of the values over time.

### Embodiments

Embodiment 1. A computer program product comprising a computer-readable medium having computer-executable code encoded therein, which computer-executable code is configured to implement a method comprising providing an instruction for a fermentation system, wherein said computer program product is stored in a cloud-based system, wherein said computer program product is operably connected over said cloud-based system to said fermentation system.

Embodiment 2. The computer program product of embodiment 1, wherein said instruction comprises an experimental design module, wherein said method further comprises receiving by said experimental design module an experimental protocol from a user.

Embodiment 3. The computer program product of embodiment 2, wherein said experimental protocol comprises i) a microbial strain for use in said fermentation system; ii) a culture medium for said microbial strain; and iii) a fermentation protocol.

Embodiment 4. The computer program product of any one of embodiments 1-3, wherein said instruction comprises a protocol design module, wherein said method further comprises receiving by the protocol design module a fermentation protocol by said user.

Embodiment 5. The computer program product of any one of embodiments 3-4, wherein said fermentation protocol comprises i) said microbial strain for use in said fermentation system; ii) said culture medium for said microbial strain; iii) a duration for said fermentation protocol; and iv) a set-point for a biological parameter of said fermentation system.

Embodiment 6. The computer program product of any one of embodiments 2-5, wherein said instruction comprises a monitoring module, wherein said monitoring module monitors said experimental protocol, wherein said monitoring module assesses the suitability of said experimental protocol for said fermentation system.

Embodiment 7. The computer program product of any one of embodiments 2-6, wherein said instruction comprises a display module, wherein said display module displays to said user data obtained from said fermentation system based on said experimental protocol.

Embodiment 8. The computer program product of any one of embodiments 1-7, wherein said fermentation system comprises a bioreactor.

Embodiment 9. The computer program product of any one of embodiments 1-8, wherein said fermentation system comprises a plurality of bioreactors.

Embodiment 10. The computer program product of any one of embodiments 3-9, wherein the microbial strain is provided by the user.

Embodiment 11. The computer program product of any one of embodiments 3-10, wherein the culture medium is provided by the user.

Embodiment 12. The computer program product of any one of embodiments 3-11, wherein the duration for the fermentation protocol is from about 24 hours to about one week.

Embodiment 13. The computer program product of any one of embodiments 5-12, wherein the biological parameter is pH.

Embodiment 14. The computer program product of any one of embodiments 5-12, wherein the biological parameter is temperature.

Embodiment 15. The computer program product of any one of embodiments 5-12, wherein the biological parameter is an optical density of the culture medium.

Embodiment 16. The computer program product of any one of embodiments 5-12, wherein the biological parameter is dissolved oxygen.

Embodiment 17. The computer program product of any one of embodiments 7-16, wherein the display module displays a status of the experimental protocol to the user.

Embodiment 18. The computer program product of any one of embodiments 7-17, wherein the display module displays progress of the experimental protocol to the user.

Embodiment 19. The computer program product of any one of embodiments 7-18, wherein the display module displays data associated with a biological parameter of said fermentation system.

Embodiment 20. The computer program product of any one of embodiments 1-19, wherein said user adjusts a biological parameter of said fermentation system in real-time.

Embodiment 21. A method for controlling a fermentation system, the method comprising: a computing system, wherein said computing system comprises a digital computer with cloud access to a computing platform over a network, wherein said digital computer comprises a computer processor and a computer memory comprising a computer program executable by said computer processor to generate an instruction for said fermentation system, wherein the computing platform is operably linked to said fermentation system, wherein said computing platform is configured to execute said instruction in response to a user input.

Embodiment 22. The method of embodiment 21, wherein said instruction comprises an experimental design module, wherein said method further comprises receiving by said experimental design module an experimental protocol from a user.

Embodiment 23. The method of embodiment 22, wherein said experimental protocol comprises i) a microbial strain for use in said fermentation system; ii) a culture medium for said microbial strain; and iii) a fermentation protocol.

Embodiment 24. The method of any one of embodiments 21-23, wherein said instruction comprises a protocol design module, wherein said method further comprises receiving by the protocol design module a fermentation protocol by said user.

Embodiment 25. The method of embodiment 24, wherein said fermentation protocol comprises i) said microbial strain for use in said fermentation system; ii) said culture medium for said microbial strain; iii) a duration for said fermentation protocol; and iv) a set-point for a biological parameter of said fermentation system.

Embodiment 26. The method of any one embodiments 22-25, wherein said instruction comprises a monitoring module, wherein said monitoring module monitors said experimental protocol, wherein said monitoring module assesses the suitability of said experimental protocol for said fermentation system.

Embodiment 27. The method of any one of embodiments 22-26, wherein said instruction comprises a display module, wherein said display module displays to said user data obtained from said fermentation system based on said experimental protocol.

Embodiment 28. The method of any one of embodiments 21-27, wherein said fermentation system comprises a bioreactor.

Embodiment 29. The method of any one of embodiments 21-28, wherein said fermentation system comprises a plurality of bioreactors.

Embodiment 30. The method of any one of embodiments 23-29, wherein the microbial strain is provided by the user.

Embodiment 31. The method of any one of embodiments 23-30, wherein the culture medium is provided by the user.

Embodiment 32. The method of any one of embodiments 25-31, wherein the duration for the fermentation protocol is from about 24 hours to about one week.

Embodiment 33. The method of any one of embodiments 25-32, wherein the biological parameter is pH.

Embodiment 34. The method of any one of embodiments 25-32, wherein the biological parameter is temperature.

Embodiment 35. The method of any one of embodiments 25-32, wherein the biological parameter is an optical density of the culture medium.

Embodiment 36. The method of any one of embodiments 25-32, wherein the biological parameter is dissolved oxygen.

Embodiment 37. The method of any one of embodiments 27-36, wherein the display module displays a status of the experimental protocol to the user.

Embodiment 38. The method of any one of embodiments 27-37, wherein the display module displays progress of the experimental protocol to the user.

Embodiment 39. The method of any one of embodiments 27-38, wherein the display module displays data associated with a biological parameter of said fermentation system.

Embodiment 40. The method of any one of embodiments 21-39, wherein said user adjusts a biological parameter of said fermentation system in real-time.

Embodiment 100. A computer program product comprising a computer-readable medium having computer-executable code encoded therein, which computer-executable code is configured to implement a method comprising providing an instruction for a fermentation system, wherein said computer program product is stored in a cloud-based system, wherein said computer program product is operably connected over said cloud-based system to said fermentation system, wherein the fermentation system comprises: a) a plurality of bioreactors configured to receive a fermentation agent, wherein at least one of the bioreactors of said plurality of bioreactors is removable and capable of having a different configuration from at least one other bioreactor of said plurality of bioreactors; and b) a first robotic component configured to provide said fermentation agent to at least one of said plurality of bioreactors.

Embodiment 101. The computer program product of embodiment 100, wherein said instruction comprises an experimental design module, wherein said method further comprises receiving by said experimental design module an experimental protocol from a user.

Embodiment 102. The computer program product of embodiment 101, wherein said experimental protocol comprises i) a fermentation agent for use in said fermentation system; ii) a culture medium for said fermentation agent; and iii) a fermentation protocol.

Embodiment 103. The computer program product of any one of embodiments 100-102, wherein said instruction further comprises a protocol design module, wherein said method further comprises receiving by the protocol design module a fermentation protocol by said user.

Embodiment 104. The computer program product of embodiment 103, wherein said fermentation protocol comprises i) said fermentation agent for use in said fermentation system; ii) said culture medium for said fermentation agent; iii) a duration for said fermentation protocol; and iv) a set-point for a biological parameter of said fermentation system.

Embodiment 105. The computer program product of any one of embodiments 103-104, wherein said instruction further comprises a planning module, wherein said method further comprises receiving by the planning module a fermentation system protocol by said user, wherein said fermentation system protocol comprises said experimental protocol and said fermentation protocol, and wherein said fermentation system protocol provides a direction for movement of said first robotic component.

Embodiment 106. The computer program product of embodiment 105, wherein said direction for movement of said first robotic component comprises directing said first robotic component to move said fermentation agent from a sample tube to at least one of said plurality of bioreactors.

Embodiment 107. The computer program product of embodiment 105, wherein said direction for movement of said first robotic component comprises directing said first robotic component to move a sample tube to a cold storage container.

Embodiment 108. The computer program product of embodiment 105, wherein said direction for movement of said first robotic component comprises directing said first robotic component to move a fermentable substrate of at least one of said plurality of bioreactors.

Embodiment 109. The computer program product of embodiment 105, wherein said instruction further comprises a configuration module, wherein said configuration module performs configuration of at least one of said plurality of bioreactors in response to said experimental protocol.

Embodiment 110. The computer program product of any one of embodiments 101-109, wherein said instruction comprises a monitoring module, wherein said monitoring module monitors said experimental protocol, wherein said monitoring module assesses the suitability of said experimental protocol for said fermentation system.

Embodiment 111. The computer program product of any one of embodiments 101-110, wherein said instruction comprises a display module, wherein said display module displays to said user data obtained from said fermentation system based on said experimental protocol.

Embodiment 112. The computer program product of any one of embodiments 100-111, wherein said first robotic component is a robotic arm.

Embodiment 113. The computer program product of any one of embodiments 100-111, wherein said first robotic component is a gantry.

Embodiment 114. The computer program product of any one of embodiments 100-113, said fermentation system further comprising a second robotic component, wherein said second robotic component is configured to aid in sample handling.

Embodiment 115. The computer program product of embodiment 114, wherein said second robotic component is a robotic arm.

Embodiment 116. The computer program product of embodiment 114, wherein said second robotic component is a gantry.

Embodiment 117. The computer program product of any one of embodiments 100-116, wherein said fermentation agent is yeast.

Embodiment 118. The computer program product of any one of embodiments 100-116, wherein said fermentation agent is a bacterium.

Embodiment 119. The computer program product of any one of embodiments 100-116, wherein said fermentation agent is an alga.

Embodiment 120. The computer program product of any one of embodiments 100-116, wherein said fermentation agent is a fungus.

Embodiment 121. The computer program product of any one of embodiments 100-116, wherein said fermentation agent is a mammalian cell.

Embodiment 122. The computer program product of any one of embodiments 100-116, wherein said fermentation agent is an animal cell.

Embodiment 123. The computer program product of any one of embodiments 100-116, wherein said fermentation agent is an insect cell.

Embodiment 124. The computer program product of any one of embodiments 104-123, wherein said duration for the fermentation protocol is from about 24 hours to about one week.

Embodiment 125. The computer program product of any one of embodiments 104-124, wherein said biological parameter is pH.

Embodiment 126. The computer program product of any one of embodiments 104-124, wherein said biological parameter is temperature.

Embodiment 127. The computer program product of any one of embodiments 104-124, wherein said biological parameter is an optical density of said culture medium.

Embodiment 128. The computer program product of any one of embodiments 104-124, wherein said biological parameter is dissolved oxygen in said culture medium.

Embodiment 129. The computer program product of any one embodiments 111-128, wherein said display module displays a status of said experimental protocol to said user.

Embodiment 130. The computer program product of any one of embodiments 111-129, wherein said display module displays progress of said experimental protocol to said user.

Embodiment 131. The computer program product of any one of embodiments 111-130, wherein said display module displays data associated with a biological parameter of said fermentation system.

Embodiment 132. The computer program product of embodiment 104, wherein said user adjusts said set-point of said biological parameter of said fermentation protocol in real-time.

Embodiment 133. A method for controlling a fermentation system, the method comprising: a) receiving by a computer program executable by a processor of a computer system an instruction for said fermentation system by a user, wherein said computer system is operably linked to said fermentation system, wherein said computer program is stored in a cloud-based system; and b) executing by said computer program executable by said processor of said computer system said instruction in response to input from said user; wherein said fermentation system comprises: i) a plurality of bioreactors configured to receive a fermentation agent, wherein at least one of the bioreactors of said plurality of bioreactors is removable and capable of having a different configuration from at least one other bioreactor of said plurality of bioreactors; and ii) a first robotic component configured to provide said fermentation agent to at least one of said plurality of bioreactors.

Embodiment 134. The method of embodiment 133, wherein said instruction comprises an experimental protocol provided by said user, wherein said experimental protocol comprises i) a fermentation agent for use in said fermentation system; ii) a culture medium for said fermentation agent; and iii) a fermentation protocol.

Embodiment 135. The method of embodiment 134, the method comprising receiving by said computer program an experimental protocol provided by said user.

Embodiment 136. The method of any one of embodiments 133-135, wherein said instruction comprises a fermentation protocol provided by said user wherein said fermentation protocol comprises i) said fermentation agent for use in said fermentation system; ii) said culture medium for said fermentation agent; iii) a duration for said fermentation protocol; and iv) a set-point for a biological parameter of said fermentation system.

Embodiment 137. The method of embodiment 136, the method comprising receiving by said computer program a fermentation protocol provided by said user.

Embodiment 138. The method of any one of embodiments 136-137, wherein said instruction further comprises a fermentation system protocol provided by said user, wherein said fermentation system protocol comprises said experimental protocol and said fermentation protocol, and the method further comprises directing movement of said first robotic component based on said fermentation system protocol.

Embodiment 139. The method of embodiment 138, wherein said directing movement of said first robotic component comprises directing said first robotic component to move said fermentation agent from a sample tube to at least one of said plurality of bioreactors.

Embodiment 140. The method of embodiment 138, wherein said directing movement of said first robotic component comprises directing said first robotic component to move a sample tube to a cold storage container.

Embodiment 141. The method of embodiment 138, wherein said directing movement of said first robotic component comprises directing said first robotic component to move a fermentable substrate of at least one of said plurality of bioreactors.

Embodiment 142. The method of any one of embodiments 134-141, wherein said instruction further comprises configuring by said computer program at least one of said plurality of bioreactors in response to said experimental protocol.

Embodiment 143. The method of any one of embodiments 134-142, wherein said instruction further comprises monitoring by said computer program said experimental protocol, wherein said monitoring assesses the suitability of said experimental protocol for said fermentation system.

Embodiment 144. The method of any one of embodiments 134-143, wherein said instruction further comprises displaying to said user data obtained from said fermentation system based on said experimental protocol.

Embodiment 145. The method of any one of embodiments 133-144, wherein said fermentation agent is yeast.

Embodiment 146. The method of any one of embodiments 133-144, wherein said fermentation agent is a bacterium.

Embodiment 147. The method of any one of embodiments 133-144, wherein said fermentation agent is an alga.

Embodiment 148. The method of any one of embodiments 133-144, wherein said fermentation agent is a fungus.

Embodiment 149. The method any one of embodiments 133-144, wherein said fermentation agent is a mammalian cell.

Embodiment 150. The method of any one of embodiments 133-144, wherein said fermentation agent is an animal cell.

Embodiment 151. The method of any one of embodiments 133-144, wherein said fermentation agent is an insect cell.

Embodiment 152. The method of any one of embodiments 136-151, wherein said duration for the fermentation protocol is from about 24 hours to about one week.

Embodiment 153. The method of any one of embodiments 133-152, wherein said first robotic component is a robotic arm.

Embodiment 154. The method of any one of embodiments 133-152, wherein said first robotic component is a gantry.

Embodiment 155. The method of any one of embodiments 133-154, said fermentation system further comprising a second robotic component, wherein said second robotic component is configured to aid in sample handling.

Embodiment 156. The method embodiment 155, wherein said second robotic component is a robotic arm.

Embodiment 157. The method of embodiment 155, wherein said second robotic component is a gantry.

Embodiment 158. The method of any one of embodiments 136-157, wherein said biological parameter is pH.

Embodiment 159. The method of any one of embodiments 136-157, wherein said biological parameter is temperature.

Embodiment 160. The method of any one of embodiments 136-157, wherein said biological parameter is an optical density of said culture medium.

Embodiment 161. The method of any one of embodiments 136-157, wherein said biological parameter is dissolved oxygen.

Embodiment 162. The method of any one of embodiments 144-161, wherein said displaying displays a status of said experimental protocol to said user.

Embodiment 163. The method of any one of embodiments 144-162, wherein said displaying displays progress of said experimental protocol to said user.

Embodiment 164. The method of any one of embodiments 144-163, wherein said displaying displays data associated with a biological parameter of said fermentation system.

Embodiment 165. The method of any one of embodiments 144-164, wherein said user adjusts said set-point for said biological parameter of said fermentation system in real-time.

Embodiment 166. A system for controlling a fermentation system, the system comprising a computing system, wherein said computing system comprises a digital computer with access to a computing platform, wherein said digital computer comprises a computer processor and a computer memory comprising a computer program executable by said computer processor to generate an instruction for a fermentation system, wherein said computer program is stored in a cloud-based system, wherein said computer platform is operably linked to said fermentation system, wherein said fermentation system comprises: a) a plurality of bioreactors configured to receive a fermentation agent, wherein at least one of the bioreactors of said plurality of bioreactors is removable and capable of having a different configuration from at least one other bioreactor of said plurality of bioreactors; and b) a first robotic component configured to provide said fermentation agent to at least one of the plurality of bioreactors.

Embodiment 167. The system of embodiment 166, wherein said instruction comprises an experimental design module, wherein said method further comprises receiving by said experimental design module an experimental protocol from a user.

Embodiment 168. The system of embodiment 167, wherein said experimental protocol comprises i) a fermentation agent for use in said fermentation system; ii) a culture medium for said fermentation agent; and iii) a fermentation protocol.

Embodiment 169. The system of any one of embodiments 166-168, wherein said instruction further comprises a protocol design module, wherein said method further comprises receiving by the protocol design module a fermentation protocol by said user.

Embodiment 170. The system of embodiment 169, wherein said fermentation protocol comprises i) said fermentation agent for use in said fermentation system; ii) said culture medium for said fermentation agent; iii) a duration for said fermentation protocol; and iv) a set-point for a biological parameter of said fermentation system.

Embodiment 171. The system of any one of embodiments 169-170, wherein said instruction further comprises a planning module, wherein said method further comprises receiving by the planning module a fermentation system protocol by said user, wherein said fermentation system protocol comprises said experimental protocol and said fermentation protocol, and wherein said fermentation system protocol provides a direction for movement of said first robotic component.

Embodiment 172. The system of embodiment 171, wherein said direction for movement of said first robotic component comprises directing said first robotic component to move said fermentation agent from a sample tube to at least one of said plurality of bioreactors.

Embodiment 173. The system of embodiment 171, wherein said direction for movement of said first robotic component comprises directing said first robotic component to move a sample tube to a cold storage container.

Embodiment 174. The system of embodiment 171, wherein said direction for movement of said first robotic component comprises directing said first robotic component to move a fermentable substrate of at least one of said plurality of bioreactors.

Embodiment 175. The system of any one of embodiments 167-174, wherein said instruction further comprises a configuration module, wherein said configuration module performs configuration of at least one of said plurality of bioreactors in response to said experimental protocol.

Embodiment 176. The system of any one of embodiments 167-175, wherein said instruction comprises a monitoring module, wherein said monitoring module monitors said experimental protocol, wherein said monitoring module assesses the suitability of said experimental protocol for said fermentation system.

Embodiment 177. The system of any one of embodiments 167-176, wherein said instruction comprises a display module, wherein said display module displays to said user data obtained from said fermentation system based on said experimental protocol.

Embodiment 178. The system of any one of embodiments 166-177, wherein said first robotic component is a robotic arm.

Embodiment 179. The system of any one of embodiments 166-177, wherein said first robotic component is a gantry.

Embodiment 180. The system of any one of embodiments 166-179, said fermentation system further comprising a second robotic component, wherein said second robotic component is configured to aid in sample handling.

Embodiment 181. The system of embodiment 180, wherein said second robotic component is a robotic arm.

Embodiment 182. The system of embodiment 180, wherein said second robotic component is a gantry.

Embodiment 183. The system of any one of embodiments 166-182, wherein said fermentation agent is yeast.

Embodiment 184. The system of any one of embodiments 166-182, wherein said fermentation agent is a bacterium.

Embodiment 185. The system of any one of embodiments 166-182, wherein said fermentation agent is an alga.

Embodiment 186. The system of any one of embodiments 166-182, wherein said fermentation agent is a fungus.

Embodiment 187. The system of any one of embodiments 166-182, wherein said fermentation agent is a mammalian cell.

Embodiment 188. The system of any one of embodiments 166-182, wherein said fermentation agent is an animal cell.

Embodiment 189. The system of any one of embodiments 166-182, wherein said fermentation agent is an insect cell.

Embodiment 190. The system of any one of embodiments 170-189, wherein said duration for the fermentation protocol is from about 24 hours to about one week.

Embodiment 191. The system of any one of embodiments 170-190, wherein said biological parameter is pH.

Embodiment 192. The system of any one of embodiments 170-190, wherein said biological parameter is temperature.

Embodiment 193. The system of any one of embodiments 170-190, wherein said biological parameter is an optical density of the culture medium.

Embodiment 194. The system of any one of embodiments 170-190, wherein said biological parameter is dissolved oxygen.

Embodiment 195. The system of any one of embodiments 177-194, wherein said display module displays a status of said experimental protocol to said user.

Embodiment 196. The system of any one of embodiments 177-194, wherein said display module displays progress of said experimental protocol to said user.

Embodiment 197. The system of any one of embodiments 177-194, wherein said display module displays data associated with a biological parameter of said fermentation system.

Embodiment 198. The system of any one of embodiments 170-197, wherein said user adjusts said set-point of said biological parameter of said fermentation protocol in real-time.

Embodiment 200. A system for controlling an automated fermentation system, the system comprising: a computing system, wherein said computing system comprises a digital computer with access to a computing platform over a network, wherein said digital computer comprises a computer processor and a computer memory comprising a computer program executable by said computer processor to generate an instruction for said fermentation system, wherein the computing platform is operably linked to said automated fermentation system, wherein said computing platform is configured to execute said instruction in response to a user input, wherein said instruction further comprises a planning module, wherein said system further comprises receiving by said planning module a fermentation system protocol from a user; wherein the automated fermentation system comprises: a) a plurality of bioreactors configured to receive a fermenting agent, wherein at least one of the bioreactors of said plurality of bioreactors is removable and capable of having a different configuration from at least one other bioreactor of said plurality of bioreactors; and b) a first robotic component configured to provide said fermenting agent to at least one of the plurality of bioreactors.

Embodiment 201. The system of embodiment 200, wherein the automated fermentation system further comprises an automated seed train preparation station.

Embodiment 202. The system of any one of embodiments 200-201, wherein said fermentation system protocol comprises a direction for movement of said first robotic component.

Embodiment 203. The system of any one of embodiments 200-202, wherein said instruction comprises an experimental design module, wherein said system further comprises receiving by said experimental design module an experimental protocol from a user.

Embodiment 204. The system of embodiment 203, wherein said experimental protocol comprises i) a microbial strain for use in said fermentation system; ii) a culture medium for said microbial strain; and iii) a fermentation protocol.

Embodiment 205. The system of any one of embodiments 200-204, wherein said instruction comprises a protocol design module, wherein said system further comprises receiving by the protocol design module a fermentation protocol by a user.

Embodiment 206. The system of embodiment 205, wherein said fermentation protocol comprises i) said microbial strain for use in said fermentation system; ii) said culture medium for said microbial strain; iii) a duration for said fermentation protocol; and iv) a set-point for a biological parameter of said fermentation system.

Embodiment 207. The system of embodiment 203, wherein said instruction comprises a monitoring module, wherein said monitoring module monitors said experimental protocol, wherein said monitoring module assesses the suitability of said experimental protocol for said automated fermentation system.

Embodiment 208. The system of any one of embodiments 200-207, wherein said instruction comprises a display module, wherein said display module displays to said user data obtained from said automated fermentation system based on said experimental protocol.

Embodiment 209. The system of any one of embodiments 200-208, wherein said automated fermentation system comprises a bioreactor.

Embodiment 210. The system of any one of embodiments 200-209, wherein said automated fermentation system comprises a plurality of bioreactors.

Embodiment 211. The system of any one of embodiments 200-210, wherein the first robotic component is a robotic arm.

Embodiment 212. The system of any one of embodiments 200-211, wherein the first robotic component is a gantry.

Embodiment 213. The system of any one of embodiments 200-212, further comprising a second robotic component, wherein the second robotic component is configured to aid in sample handling.

Embodiment 214. The system of embodiment 213, wherein the second robotic component is a robotic arm.

Embodiment 215. The system of embodiment 213, wherein the second robotic component is a gantry.

1.-66. (canceled)

67. A system for controlling a fermentation system, the system comprising a computing system, wherein said computing system comprises a digital computer with access to a computing platform, wherein said digital computer comprises a computer processor and a computer memory comprising a computer program executable by said computer processor to generate an instruction for a fermentation system, wherein said computer program is stored in a cloud-based system, wherein said computer platform is operably linked to said fermentation system,
wherein said fermentation system comprises:
a) a plurality of bioreactors configured to receive a fermentation agent, wherein at least one of the bioreactors of said plurality of bioreactors is removable and capable of having a different configuration from at least one other bioreactor of said plurality of bioreactors; and
b) a first robotic component configured to provide said fermentation agent to at least one of the plurality of bioreactors.

68. The system of claim 67, wherein said instruction comprises an experimental design module, wherein said method further comprises receiving by said experimental design module an experimental protocol from a user.

69. The system of claim 68, wherein said experimental protocol comprises i) a fermentation agent for use in said fermentation system; ii) a culture medium for said fermentation agent; and iii) a fermentation protocol.

70. The system of claim 68, wherein said instruction further comprises a protocol design module, wherein said method further comprises receiving by the protocol design module a fermentation protocol by said user.

71. The system of claim 70, wherein said fermentation protocol comprises i) said fermentation agent for use in said fermentation system; ii) said culture medium for said fermentation agent; iii) a duration for said fermentation protocol; and iv) a set-point for a biological parameter of said fermentation system.

72. The system of claim 70, wherein said instruction further comprises a planning module, wherein said method further comprises receiving by the planning module a fermentation system protocol by said user, wherein said fermentation system protocol comprises said experimental protocol and said fermentation protocol, and wherein said fermentation system protocol provides a direction for movement of said first robotic component.

73. The system of claim 72, wherein said direction for movement of said first robotic component comprises directing said first robotic component to move said fermentation agent from a sample tube to at least one of said plurality of bioreactors, directing said first robotic component to move a sample tube to a cold storage container, or directing said first robotic component to move a fermentable substrate of at least one of said plurality of bioreactors.

74. (canceled)

75. (canceled)

76. The system of claim 72, wherein said instruction further comprises a configuration module, wherein said configuration module performs configuration of at least one of said plurality of bioreactors in response to said experimental protocol.

77. The system of claim 68, wherein said instruction comprises a monitoring module, wherein said monitoring module monitors said experimental protocol, wherein said monitoring module assesses the suitability of said experimental protocol for said fermentation system.

78. The system of claim 68, wherein said instruction comprises a display module, wherein said display module displays to said user data obtained from said fermentation system based on said experimental protocol.

79. The system of claim 67, wherein said first robotic component is a robotic arm or gantry.

80. (canceled)

81. The system of claim 67, said fermentation system further comprising a second robotic component configured to aid in sample handling, wherein said second robotic component is a robotic arm or gantry.

82. (canceled)

83. (canceled) .

84. The system of claim 67, wherein said fermentation agent is a yeast, a bacterium, an alga, or a fungus.

85. (canceled)

86. (canceled)

87. (canceled)

88. The system of claim 67, wherein said fermentation agent is a mammalian cell.

89. The system of claim 67, wherein said fermentation agent is an animal cell or insect cell.

90. (canceled)

91. The system of claim 71, wherein said duration for the fermentation protocol is from about 24 hours to about one week.

92. The system of claim 71, wherein said biological parameter is pH, temperature, or dissolved oxygen.

93. (canceled)

94. The system of claim 71, wherein said biological parameter is an optical density of the culture medium.

95. (canceled)

96. The system of claim 78, wherein said display module displays a status of said experimental protocol to said user, a progress of said experimental protocol to said user, or data associated with a biological parameter of said fermentation system.

97. (canceled)

98. (canceled)

99. The system of claim 71, wherein said user adjusts said set-point of said biological parameter of said fermentation protocol in real-time.
